SEQUENCE LISTING (57 sequences)

SEQ ID NO: 1 - DNA, 263 bp, Homo sapiens
acaagctttttccagcaggtcaccttctgtggtttaatcagtctatcaggcctacaggtctgactcaaagttgcaatcttctctttcaccccttttctgaaatcttgaagtctggcaaatatgtatcaagaaccgttattagccgacagcctcccgcaactctttttttcctccgtttcccggacggacttcacagcaaaatcctgtcgccaagagaagcgtggcccgaagcccctgctcgcaccctgccccgcctaggcggc

SEQ ID NO: 2 - DNA, 720 bp, Homo sapiens
gtgaaacaccctcggctgggaagtcagttcgttctctcctctcctctcttcttgtttgaacatggtgcggactaaagcagacagtgttccaggcacttacagaaaagtggtggctgctcgagcccccagaaaggtgcttggttcttccacctctgccactaattcgacatcagtttcatcgaggaaagctgaaaataaatatgcaggagggaaccccgtttgcgtgcgcccaactcccaagtggcaaaaaggaattggagaattctttaggttgtcccctaaagattctgaaaaagagaatcagattcctgaagaggcaggaagcagtggcttaggaaaagcaaagagaaaagcatgtcctttgcaacctgatcacacaaatgatgaaaaagaatagaactttctcattcatctttgaataacgtctccttgtttaccctggtattctagaatgtaaatttacataaatgtgtttgttccaattagctttgttgaacaggcatttaattaaaaaatttaggtttaaatttagatgttcaaaagtagttgtgaaatttgagaatttgtaagactaattatggtaacttagcttagtattcaatataatgcattgtttggtttcttttaccaaattaagtgtctagttcttgctaaaatcaagtcattgcattgtgttctaattacaagtatgttgtatttgagatttgcttagattgttgt

SEQ ID NO: 3 - DNA, 482 bp, Homo sapiens
  modified_base (448)..(465): N = A, C, G or T/U
acttgcggcacttgtggtgtaggctgaagagatgggggctgttgcagctgatggggggtgtgatgaggcatctgttgttttccttgtgagtaaagatccaggatttggtggcagatgtcttccaaaacgtcgaccgggacatcttgaacaaactgctcccaccatctcctatacatgggtttggaggtccattcttgtatttcaaatttgcacaaacgtcctgcgagatacatcactgctactgctatgatctctggttcccactgcagtgacaaggtggtgcagagactgtcatttacaaatgtccatgccatttgaaccaacttttgaattttgtttttatcacctttgagttgctttgcatattttagtaggaactggtatggatgttctacctgtaaatcaaacttgatggctgcagtaagattctctccagaaccattacttnctcctttgggtcatctncaaactggccaaattgt

SEQ ID NO: 4 - DNA, 553 bp, Homo sapiens
acaagggggccctcaaacatgatagcaggaactacgcgtggaacatctgtctaatgtagcatccttacttcctgcctcagttaccaaagaaacctctgatgcaggtctgctgccccgacggggccaggactccacagcgctttctcagtcacaagccatgatgaattggtgactcagacgctttgtgctttttcctttgcttcttgagaccggggtgtgtgtggctcagcttccacggcgtgtttggttcggtccatgtgtgtgcgtgtgtatacttgaagagaactgtcgtgtctgatttgcactattggaggaggactaaagttgcctgacaactttatgtgttatgccagaactctgagggcaaactgctgaaaaacaaagggtttaaggatgacatttctgaccatttgtgtgtttggtggtggtactggttttgttttttttaatgtagacaatacagctttggaaggggaagtctcatacaggttataggtctttctctctctagatttcaggtgcttgcaactggactgcagactctaccaatcac

SEQ ID NO: 5 - DNA, 287 bp, Homo sapiens
  modified_base (205)..(283): N = A, C, G, or T/U
acagccgccgcttccagaccattgacatcgagcctgacatcgaagccctgctgtctcaagggcccagctgtgcctagggcgcccctcctaccccggctgcttggcagttgcagtgctgctgtctcgggggggttttcatctatgagggtgtttcctctaaacctacgagggaggaacacctgatcttacagaaaataccacctcnagatgggtgctggtcctgttgatcccagtctctgccagaccaaggcgagtttccccactaataaagtgccgggtgtcngcag

SEQ ID NO: 6 - DNA, 615 bp, Homo sapiens
  modified_base (428)..(490): N = A, C, G or T/U
ctgaactctcgctttctttttaatcccctgcatcggatcaccggcgtgccccaccatgtcagacgcagccgtagacaccagctccgaaatcaccaccaaggacttaaaggagaagaaggaagttgtggaagaggcagaaaatggaagagacgcccctgctaacgggaatgctaatgaggaaaatggggagcaggaggctgacaatgaggtagacgaagaagaggaagaaggtggggaggaagaggaggaggaagaagaaggtgatggtgaggaagaggatggagatgaagatgaggaagctgagtcagctacgggcaagcgggcagctgaagatgatgaggatgaccatgtcgatccaagaagcagaagaccgacgaggatgactagacagcaaaaaaggaaaagttaaactaaaaaaaaaaggccgncgtgacctattcaccctccctttccgtctcagaatctaaacgtggtcccttcgagtagagangccccccgcccaccgtgggcagtgcccccgcagatgacacgcgcttttcacccccaacccaaccatgagaatttccacagggggaggaaaaaaaaacccaaactttcaagggccctgcttttttt

SEQ ID NO: 7 - DNA, 629 bp, Homo sapiens
  modified_base (212)..(491): N = A, C, G, or T/U
acagagagataaaggatgctaaagagcaggtaagccctttggaaacaacattggaaaagttccagcaagaaaaagaagaattaatcaacaaaaaaatacaagcaacaaaatagcacaggataaactgaatgatattaaagagaaggttaaaaatattcatggctatatgaaagacattgagaattatattcaagatgggaaagacgactatnagaagcaaaaagaaactgaacttaataaagtaatagctcaactaagtgaatgcgagaaacacaaagaaaagataaatgaagatatgagactcatgagacaagatattgatacacagaagatacaagaaaggtggctacaagataaccttactttaagaaaaagaaatgaggaactaaaagaagttgaagaagaaagaaaacacatttgaaggaaatgggtcaaatgcaggttttgcaaatgaaaagtgacatcagaagttggaagagaacatgaccatntaaaaagaaatcataatttggcattagggcgacagaaagggtatgaagaagaaattattcattttaagaaagacttcgagaccacaatttcgggatgctgaggaaaagttttagagaaaatggatgatttggttttga

SEQ ID NO: 8 - DNA, 621 bp, Homo sapiens
acatcatctctgtcatacacagtgcagtccaccaggagacgcaatccaccaccacgaactcttcatccgatcagcacgagccattcatgtgctgaaacaccaagatctgtggaagaaatcctcagaggagcccgagtcccagtggcacccgactcgctctcctctccctcaccgacgcccctgagtcgaaataatctgctaccacctattggcacagctgaagtggaacatgtgagcactgtggggccacaaagacagatgaaaccccatggcgactctagtcgagctcaaagtgcggtggtggatgaacctaactatcagcagccacaagaaaggctccttttgcccgactttttccccaggcccaacacaactcaatcatttttgctggatacacagtatcgtcgctcatgtgcagttgagtatcctcatcaggcccgacctgcaggggatctgcaggtcctcagttacatgggtctacaaaatctcaaagcggaggcagaccaagtctctcgaaccaggcagggaccataaggcaaatgagaagaatctatcaggctgcaggaaacacgagatttcatgaagcagtattcagtcatcaaagtgatgccagagctttgt

SEQ ID NO: 9 - DNA, 551 bp, Homo sapiens
acagaaaggtgttcttacatgaagaagggtgtgaaggctgaacaatcatggatttttctgatcaattgtgctttaggaaattattgacagttttgcacaggttcttgaaaacgttatttataatgaaatcaactaaaactatttttgctataagttctataaggtgcataaaacccttaaattcatctagtagctgttcccccgaacaggtttattttagtaaaaaaaaaaaacaaaaaacaaaaacaaaagatttttatcaaatgttatgatgcaaaaaaagaaaaagaaaaaaaaaagaaaagaaaacttcaattttctgggtatgcacaaagaccatgaagacttatccaagtgcatgaccggatttttgtggttttgttcattttgtgtttaatttgtgtttttttttccagctgtatgaaatgggctttctgaagtttaaatagtccgacttcacccatggtgttctgtgcttgcagtgcgagtgttgctgtaattcatgttgccgtcaggtctcttttcttaactttctgctttctttcaacgta

SEQ ID NO: 10 - DNA, 511 bp, Homo sapiens
acttgaactggcaggaaatgcatcaaaagacttaaaggtaaagcgtattacccctcgtcacttgcaacttgctattcgtggagatgaagaattggattctctcatcaaggctacaattgctggtggtggtgtcattccacacatccacaaatctctgattgggaagaaaggacaacagaagactgtctaaaggatgcctggattccttgttatctcaggactctaaatactctaacagctgtccagtgttggtgattccagtggactgtatctctgtgaaaaacacaattttgcctttttgtaattctatttgagcaagttggaagtttaattagctttccaaccaaccaaatttctgcattcgagtcttaaccatatttaagtgttactgtggcttcaaagaagctattgattctgaagtagtgggttttgattgagttgactgtttttaaaaaactgtttggattttaattgtgatgcagaagttatagtaacaaacatttggttttgt

SEQ ID NO: 11 - DNA, 507 bp, Homo sapiens
accagtatctcagagcctccgtctcctatcagtccgtatgcttcagaaagctgtggaacgctacctcttcctttgagaccttgtggagaagggtctgaaatggtaggcaaagagaatagttccccagagaataaaaactggttgttggccatggcagccaaacggaaggctgagaatccatctccacgaagtccgtcatcccagacacccaattccaggagacagagcggaaagacattgccaagcccggtcaccatcacgcccagctccatgaggaaaatctgcacatacttccatagaaagtcccaggaggacttctgtggtcctgaacactcaacagaattatagattctaatctgagtgagttactgagctttggtccactaaaacaagctgagctttggtccactaaaacaagatgaaaaatacaagagtgactctataactctggtctttaagaaagctgccttttcatttttagacaaaatcttttcaacgctgaaatgt

SEQ ID NO: 12 - DNA, 588 bp, Homo sapiens
  modified_base (394)..(494): N = A, C, G or T/U
actcagaagtcatatgttatttacaattgggtttgtgtgggatgggaagtaaggcggatgagccagtgcttttgcaatgaagatgcaatagtcattgtcctctcccactgtctcctctttcctcaccccatggcagctttcatgacccattcccaaagggtccaccgagtcctgaactcagcttcatcaccaacattcctcgccttcagttgaattcaacactgttaagggagtagaggcaaagacttggtcagggagagggtgggaaacacagaacaaactctctcggcacaacccaagttcagagacgaggcctcctcaaatgaggaagatgatgccctcagacaccatgacctgattgtcatcctgcatcttgctcagagcagcctggatntcaactgaagagaaagctctttgctgtcccggttgatggattctgtgaggcgaatcatgccgattgactgcgcatgagcttcccggaacacatccaaganggccacctttgaatgccttcaacctggattcactcaactccactttctgggattccttggtctccctgtgagtctgccgtctttggagtgtgta

SEQ ID NO: 13 - DNA, 695 bp, Homo sapiens
  modified_base (592): N = A, C, G or T/U
acacaaaccaggctgaacaatgccgcaaaatatctgccagtttacagtcccaaagtcccgagcatctcttacctgtgttaatccaagctgcccagctctgccgtgaaaagcagcacacaaaagcaatagagctgcttcaggaattttcagatcagcatccagaaaatgcagctgaaattaagctgaccatggcacagttgaaaatttctcaaggtaatatttctaaagcatgtctaatattgagaagcatagaggagttaaagcataaaccaggcatggtatctgcattagttaccatgtatagccatgaagaagatattgatagtgccattgaggtcttcacacaagctatccagtggtatcaaaaccatcagccaaaatctcctgctcatttgtccttgataagagaagctgcaaacttcaaactcaaatatgggcggaagaaggaggcaattagtgacctacaacagctgtggaaacaaaatccaaaagatattcacaccctggcacagcttatttctgcttactcacttgtagatccagagaaagccaaagctcttagtaaacacttgccatcgtcagatagtatgtntctaaaagtagatgttgaggctcttgaaaattctgctggtgctacatacattcggaagaagggggaaaagttactggagatagtcaccaaaggaacaaggacg

SEQ ID NO: 14 - DNA, 388 bp, Homo sapiens
  modified_base (204, 247)..(303): N = A, C, G or T/U
acaagctttttttttttttttttttttgccttaaatataagaaatttattaatattattaattgattaataattgatcactgccatatgacttggattttacatttccaccacttcttcgtgttacttctttctaccctattatatcagcgtcccatcccaagtctgacacacctggaagtattgaatgttgtattttgattgnggatgacttgtttatttggttatatcttttgaacgcctcaggngattctgtctgtctttggaggatgggccataggcttcctgccccaactccattatntgttttccacaaagatagataaacattcacttatcagactaattcagcaaagcatacacgttcttaggttcttagcaaaccaagt

SEQ ID NO: 15 - DNA, 499 bp, Homo sapiens
  modified_base (336, 433)..(452): N = A, C, G or T/U
acatacaagagacatattctttgacatataaaaaatacagaaatatttacatgtatacaaaaatattaaaacagatttcagaaataaggaaaacaagcaagtttttacaaaggatactttcttgggaaatacacacaatgaaaaaaagctttagaaaggtgacaacacatttaatagactccagaagttgatatttctgttggtatcaattgagtaagaggaacataaaatgtctgaaatgctaacagatggaaaaccagacatatttaagactgtctaggtaaaattgcatgggtttcctttaccataaaattgaatcaatttttttttaccctnagtatcactatctacttttgcttttttcttaaaatatttaaatggtttgatttgattgagaatttaaactttgggtgattaaatggggaaaaatatngttaagaggttgggctatnctgtttatgttctggggggaaaaaacgctagatactacacgcgattt

SEQ ID NO: 16 - DNA, 607 bp, Homo sapiens
  modified_base (503): N = A, C, G or T/U
acaacagaatatctcgggaatggactcagaagtatgccatgtgatgctaccttaaagtcagaataacctgcattatagctggaataaactttaaattactgttccttttttgattttcttatccggctgctcccctatcagacctcatcttttttaattttattttttgtttacctccctccattcattcacatgctcatctgagaagacttaagttcttccagctttggacaataactgcttttagaaactgtaaagtagttataagagaacagttgcccaagactcagaatttttaaaaaaaaaatggagcatgtgtattatgtggccaatgtcttcactctaacttggttatgagactaaaaccattcctcactgctctaacatgctgaagaaatcatctgagggggagggagatggatgctcagttgtcacatcaaaggatacagcattattctagcagcatccattcttgtttaagccttccctgttagagatttganggtacatgatatgctttatgctcataactgatgtggctggagaattggtattgaatttatagcatcagcagaacagaaaatgtgatgtattttatgcatgtcaa

SEQ ID NO: 17 - DNA, 406 bp, Homo sapiens
acttagaaacctaaggggttctttaaataggagtcagaaaaaatatataaaaataaaaaacaaagcaagaaagaacattgtataaaaataaaatgttctttaaagatgactgccctctcttgaaagatgagtgctatattaaaagggtagatgtatctctataactcatcgtgtttatatgtaaattctctggcagatataaacccatctttgtcttcatcttctttatcaaaaatatcctccaccaaagcatcatgatgactttcattcaccaccgcaccatgtttttcaaactccttctttaaatatgctttaacctcatctttagagagtttccagtcatcattaagattcatttcttggaatgattcatgggatcttggtccatttcgaatctccaggagat

SEQ ID NO: 18 - DNA, 498 bp, Homo sapiens
accttaaatcataaattctgtagtttgtatagtagtcttataaaaatatagttagctctcaaatgtttaatgccacttaagtcagttaaagtgcagattgtaaagcatattaggaaggtgcccagaataccaatgtctcctgcacttaacacattaatacaaagtttgccaattgttttgaatttccaaatgtattcctgaaaaaaaaagaacctaaacactatattatagacatatgttagaaaagtcctagaaatgcacccaatttccttccattttactttcctacatggattgaagtcagcccctcaaaagcttttcggctgggcatggtggttcacgcccataatactagcactttgggaggccaagtgggtggatacctgaggtcaggaattcaagaccagcctggccaagatggtgaaaccccatctctactaaaaaatacaaaaattacttggcgtggtagtgcgcacctgtaatcccagctactcggga

SEQ ID NO: 19 - DNA, 463 bp, Homo sapiens
  modified_base (117): N = A, C, G or T/U
acattttgtattccatagcattgctctgtggaaaattagctggcatccattttaaatgaaccctgctgaccttcttccattccgtggcctgaataaaaaatatcctcgactttgacnttgcaaagggactttggaattcttcgtccactctgaatctcttcattcttctctcatgcaggtgaggaacgagctgcagcaacggatgcacaactgacgacacaacatttgacttgcccaacttctgtgtcttcgaataatgaaataagaaccttttagtattatctttttcatcttgttcctgaggctttggtagcattcccataatcataaagcaaagctcctccagtgcgttggatgcctgaggctgagaatcaatggacagaaaagacgaacaagtatcatctatctcattccacaactgtagctgttgttcaggctgtaatccttgaggtaatattggagc

SEQ ID NO: 20 - DNA, 700 bp, Homo sapiens
  modified_base (125): N = A, C, G or T/U
acatccataatcagccaccatttaacccttccttgttctaaaacaaaaaccaaagggcgctggttggtagggtgaggtgggggagtattttaatttttggaatttgggaagcagacagctttacntttgtaaggttggaacagcagcactatacatgaaatataaaccaaaaacctttactgtttctaaatttcctagattgctattatttggttgtaagttgagtattccacagaaagtggtaattatctcttctctcttcctccattagaaaattaggtaaataatggattcctataatgggagcatcaccacttattaaaacacacatagaatgatgaattaaaaaagttttctaggattgtcttttattctgccacatttattgataaacagtgaaggaatttttaaaaaatttttaagaattgtttgtcacgtcatttttagaaatgttctacctgtatatggtaatgtccagttttaaaaatattggacatcttcaatcttaaacatttctatttagctgattggttctcacatatacttctaaaagaaacttttatgttataagagttactttttggataagatttattaatctcagttacctactattctgacattttaggaaggaggtaattgtttttaatgatggataaacttgtgctggtgttttggatcttatgatgctgagcatgtc

SEQ ID NO: 21 - DNA, 692 bp, Homo sapiens
cactttgcctctcggaaccagccttccgactcctcaccttggtctgccgggaagcgcaacaggaaaggagagaagaatggcaagggcctacggcatttctccatgaaggtctgcgagaaggtgcagaggaaagggaccacttcctacaacgaagtggcagacgagctggttgcggagttcagtgctgccgacaaccacatcttaccaaacgagtcagcttatgaccagaaaaacataagacggcgcgtctacgatgccttaaacgtgctaatggccatgaacatcatctccaaggagaagaaggagatcaagtggattggtctgcccaccaactcggctcaggaatgtcagaacttagaggtggaaagacagaggagacttgaaagaataaaacagaaacagtctcaacttcaagaacttattctacagcaaattgccttcaagaacctggtgcagagaaaccggcatgcggagcagcaggccagccggccaccgccacccaactcagtcatccacctgcccttcatcatcgtcaacaccagcaagaagacggtcatcgactgcagcatctccaatgacaaatttgagtatctgtttaattttgacaacacatttgaaatccacgatgacatagaagtgctgaagcggatgggcatggcttgcgggctggagtcggggagctgctctgccga

SEQ ID NO: 22 - DNA, 396 bp, Homo sapiens
acaacagagaatgcagcactctcctgatctaatgagctttatgatggagttgaagatgtttttggaggttgccttaaagaatagacaagagctgtatgcactacctcctcctccccagttctactcaagccttattgaagagataggaactcttggttgggataaagacaaggttttgctgtattgcccaggcgagtctcacactcctgggctgaaatgattctcctgcctcagcctcccaaaatgctgggattaacaggtgttcgctgccatgcctggcctaggatgaataaatgactttcgatttagctgctgttacttaatcatttgttttatactgtttaaaaataaagaatgttttagcagaggaaaaaaaaaaaaaaaaaaaaagcttgt

SEQ ID NO: 23 - DNA, 700 bp, Homo sapiens
aaatacctgcctgcaggcagggccatcacacccttgtgtggctccaaaatcacttttttaattaatccaatcctcagcaatgctcataacaggctaaaaggctctggggaagaaggcatttcaacagcagaaaagaggaagaagagggagaagagaaaatggagaaacaagaaataagccaacacaaaagaggggtgaagaaaagagagggtgcaaaagagaacaaagtaaaaacaaaaacaaaacaaaaaggacaaacaaatccccaaagaactctccaacggcaggtttcctggatgctccacccttccttataataaagaaggaaatggatttcttgggatttgccacttccttccctctggggctctttctccaccaccctcactccatttggctcccggctccctaggctggggtcagctgagacagcaaggaacgctggctcttggtggggcccaggacttcaaatagtccacccctggacaggacaggctttcccaaagaagttacacacagatgcttcccagccccggctcaggagggctaggatctgggccgtgaccacgtaagggcatcctggacacaatgccagggcgttgagctcacaaccagtggtctggagcttcgtgtaaactgtgatcctcaaactcctccttgagggaggaaatgggtgccttgggaggtctatggaagccta

SEQ ID NO: 24 - DNA, 524 bp, Homo sapiens
acaaagatgactataaacaagatgcagccctcggtttccatgaacagcacactattacagtaaaccaagtttatattccaccatcaagtgtggctctcccatgacttcgctttgtgatggatcattaagaatatcctcaaatccaatagtctcatcattacccctcaaaacatccagtgaaagatttgagcttgaaagaaatggaagacgctgaacctgctgcactgccttgaattccatctgtaattttagcggagcaaatagaccctgaatgtttctcagtgtggaaaaattcattttatcttggttgagctggaaatttttttctgataattcaaggggatgactaggcaaaagttcatttttcacacaagaaaaacctttccgaagaagatcatgactttcaaaaggtccacttgctgaaagttcagtaactggaatactgtcctttagctcagatccaagtcctctggcattcatcttccgcagctctgcgaacagcctctctgccccgttaccgtcag

SEQ ID NO: 25 - DNA, 698 bp, Homo sapiens
  modified_base (130): N = A, C, G or T/U
accgggtcggagattatgtctactttgagaattcctccagcaacccatacctaataagaaggatagaagaactcaacaagactgcaagtggcaacgtggaagcaaaagtagtctgcttttatagacgacngtgatatttccaacacacttataatgctcgcagataagcatgctaaagaaattgaggaagaatctgaaacaacagttgaggctgacttgaccgataagcagaaacatcagttgaaacatagggaactctttttgtcacgccagtatgaatctctgcccgcaacacatatcaggggaaagtgcagtgttgcccttctgaatgagacagaatcagtattgtcatatcttgataaggaggataccttcttctactcattggtctatgacccctcattgaaaacactattagctgacaaaggtgaaatcagagtgggacctagatatcaagcagacattccagaaatgctgttagaaggagaatcagatgagagggaacaatcaaaattggaagttaaagtttgggatccaaatagcccacttacggatcgacagattgaccagtttttagttgtagcacgtgctgttgggacattcgccagagccctggattgcagcagttctgtgaggcagcctagtttgcatatgagtgctgctgcagcttcccgagacatcaccttgtttcacgctat

SEQ ID NO: 26 - DNA, 579 bp, Homo sapiens
actactgacccaacaactgtgactagctggccacgccattcagggctggtgtggcatttatgtgtgtgtgtgtgtgtgtgtgtttttcctgtttgcccagcagtgcattgtgggttccaagagtgggtagtgtgtgtatgtgtgtgtgtcagagggagacctggcaggcacctctttgagagtagctgtggtcagagctgtttggtcagtgcattatgttgaatgaggtccaggaacccagagccacccagcagacaccactgtggcttgccagctgccaagatggagaagcatgtgcccctgtagagcgtctccccagaaccagaccccgagccactcgcttcctctgtgctgtgacaacattggtgccaggggagatggtgtttttcaaagggacctactgtagccactttaatttacaattaagagccttagtttgacttaacacttttgtaggcttttcattgtgtatttttgtgtatgtgtgcatatagcagctactctgtagcagaggtgggtagagacacttaatagtatcatgtcgcatgcagatgtcacatcggcctctgcaaaaactgt

SEQ ID NO: 27 - DNA, 653 bp, Homo sapiens
  modified_base (445): N = A, C, G or T/U
acagaaggacttaaaatgaaaaacacagtttcccatcccaccctttttaaatctaaagcccattccctagaggtaatgcttttaacaatatttattttagatcgtctggtaactttctaactttaaataatatgtttgagcaataatttcttgacttactgactttacaacatctttaataattccccattacaaaagataaggatttaacttacactatcgccactttcctttgtccatctctctccaaatgtctgatagttacatcactttttaatacatctattggtttgattttatagctttgaacaatacactaatcctctagttcttgtccattaactgaagatcttttcatccccactttgaatatataagtatctctacctttgattccacttctcttcctctaattctcaatctctttctgctttctttcccttgncagcattaattactttcaacttcagttctgaataaaaaatgaaacctttcacactttgttaataggctgatctgaacattgaatactaataaatgatatccacattattttggctatttaaatcttcttactgggagcctagtatagctaggatattttcttcctacacacccaatatcatcatgcctgtgccatagaaaaaggaaag

SEQ ID NO: 28 - DNA, 581 bp, Homo sapiens
  modified_base (23, 444)..(492): N = A, C, G or T/U
acagcaaaaaagaaactgagaancccaaactgctttcttgttaacatccacttatccaaccaatgtggaaacttcttatacttggttccattatgaagttggacaattgctgctatcacacctggcaggtaaaccaatgccaagagagtgatggaaaccattggcaagactttgttgatgaccaggattggaattttataaaaatattgttgatgggaagttgctaaagggtgaattacttccctcagaagagtgtaaagaaaagtcagagatgctataatagcagctattttaattggcaagtgccactgtggaaagagttcctgtgtgtgctgaagttctgaagggcagtcaaattcatcagcatgggctgtttggtgcaaatgcaaaagcacaggtctttttagcatgctggctctcccgtgtccttatgcaaataatcgncttcttctaaatttctcctaagcttcattttccaaagttcttcttggnttgtgatgtcttttctgctttccattaattctataaaatagtatggcttcagcccccactcttcgccttagcttgaccgtgagtctcgg

SEQ ID NO: 29 - DNA, 421 bp, Homo sapiens
  modified_base (164, 192)..(263): N = A, C, G or T/U
acagtataatcaggctaacagtatcactaaaggcctgatctggaggcaaagtggcaggcaaagctttgtctctttggtagccccctctttgtgattaaacgttttgctttgtaaggtttttgccccttattctccttttcatacatgtaaagaactttgctggngctcctttatagcctacaggtaaacttntcttctagtgagagtattgacaggagatggcttatattcccctgtttttgcaaggggaatatcctcagggntggcttcctcatttttgctgaaactactgcactgaaggtctcataggatgaggtcaactttttgttcaagagtttctgttgcggtcacccatgatggcggcctcccattggccacttttcctggcttctcgttcatcaacaggcatgaaagtggacag

SEQ ID NO: 30 - DNA, 521 bp, Homo sapiens
  modified_base (472): N = A, C, G or T/U
acatagtaagtgtatgatagatgtttgatttgtaaattacaaatataaattatcacccccatttccatttattttcttgatatatcaaaatgtgttgacttagtgattctcaatcttagctacatattagcctggcagagctttaaaaaattgatacctgggtcctattccataacaactaaatcaaaatcaccctttttttttttgtaaagctttccaggtggttctaaaggacacctggggtcaataacccattataaaatgatctgattacactggacactcttaggaagtatcaaaatagaacagttcaatttgcagctacttaatgattaattacatttcttaggttttttttgtttttttttgggttgtttgttttttgttttaaaagagacaggatcttgctttgtctcccaggctggaacacagtggtgtgatcatagctcactgcagcctgaaactccttggnttccacctcagcctcccaaagcattgggattataggcgtgagccactgc

SEQ ID NO: 31 - DNA, 515 bp, Homo sapiens
  modified_base (341)..(488): N = A, C, G or T/U
cattcttctagaaccaggttcatttttccaagttttgtagaaaaatagatgttccaagccaccttttacttaactgtctagtcttttaagaccaatcagtatgttccctggaaagatgaataagtctcatgactaattttttaaaaattctttaagacaaagaaataactttctttttttactcccaaagcacagtatctcaacagcagcagccaacatggggttttagcagcttaactttaccccctaaataaagctttgtataaaccagtgattttactacaaaaaacactgtccttgaaagaaaggagtggcagtcagacatcaatgcaaaacttggnatgattagataataaacatggcacttacaaaaggtagcttattagaatattccacttaagaagaggtacttttctgtccctccttgccccctcgaaaaacaaaaaaaaagaaaaaacatttcctttaaaaattccccttaaatgtangtttataaaatgtcagaaatgccttgt

SEQ ID NO: 32 - DNA, 466 bp, Homo sapiens
gtgaactgaaaacgtcaaccccccttgatcgtgaggagcaagctgtttatcatcttctcgtcagggccacagatggaggaggaagattctgccaagccagtattgtgctcacgctagaagatgtgaacgataatgcccccgaattctctgccgatccttatgccatcaccgtgtttgaaaacacagagccgggaacgctgctgacaagagtgcaggccacagatgccgacgcaggattaaatcggaagattttatactcactgattgactctgctgatgggcagttctccattaacgaattatctggaattattcagttagaaaaacctttggacagagaactccaggcagtatacaccctctctttgaaagctgtggatcaaggcttgccaaggaggctgactgccactggcactgtgattgtatcaagttcttgacataaatgacaacccccctgtgtttgagt

SEQ ID NO: 33 - DNA, 580 bp, Homo sapiens
acttgatcatattgatctggaagatggaatttccctgggaagagtccaaaaccactacatccacaccagcctgggcgagcaagtccagcctatacttgtcatcctcatgagtgccaatggctgccccacacagcagctgtttcttggcatctttggaggctagtgggtagtcccgattcttcttcaggtctgtccgggcaatgatggccacaagctcatcatcttcatttacaatgggcaactttcccttcttgctgcgctgcagaatttcatttgcctccttcagtgtgatgcctgcaggggctaccaccaagtcttccctctttgtcattatctcttccaagaaacagtcatgttcctcctctttgagaaaatcaatgtccctggaggagatgatgcccaccaagcggctccccatccggcctgtgtctgtgattgggataccgcagaaaccatgccgggccttggcctcaaaaacatcccgcacgcgatccttggggctgaggaccacagggtctgtgatgaatccctgttcatatttcttcactttccgaacttcattggcctggaattcaggtgt

SEQ ID NO: 34 - DNA, 464 bp, Homo sapiens
acagtcatcccactacctggctatttcattacttggtgctctagacaagctcccaagaactgactggatcttggcttgttctgtttctgtcattgctaatataatatggaaaacattgctgaaaagaacagagatggccatggatatggctaggttaggtattcatatccaaatatctgaactctaacctaatgtggatatgattctgtagcattatattaaaagctatgatgatgcaatgcaggaaataacctttcattctcccccctagaggatcacgacaggtgcttcaatgcctgccttatctatgggacagtagtgtgattctcagtgagagtgaaggcctttggggatttgagtcaggaagggaacatggctaagtgcctggaaactctgccaacagtctgcggttagattctacttgtctctggataagaaatctgtgctcagtgaacttatgtgtt

SEQ ID NO: 35 - DNA, 574 bp, Homo sapiens
ccgacaatcagatgaaaacaacttaaaagaccctgggggctccgagttcgactcgatcagcaaaaacacatgggctcctgctcctgacacatgggctcctgctcctgaccaaactgagcaagaccagaatagactgtcacagaactctgtaaatctgtctcccagcagtcacgcaaacaacttatcagtagtgacttacagtaaggggctccatgggccttaccccttcggccagtcttaaacgggtgtcagcaagaagaaaaattaacaaaagacagaaggcctgactttggggggttagggcaaggggttcctctggattgcagaccacatcgcacagacccctggctctgaccccctctcatcccggaagaagaggaagaagcagaacagactagttttgagtaaactcagtatgcatgtgtgaatgctgaatcacaggaatggtgttgaggctccaagaagaaatcatgcagccactttgggttttggtataggcatcagtctaacaagtcattaggtcactcgggaagggggaaaaagtttaaaatgggggaaaaaaagccatcttttt

SEQ ID NO: 36 - DNA, 435 bp, Homo sapiens
acaacagctaatagtttggaagccgcacagcttgacctggaagcacccctgcccccttttcagggatttttatctcgaggcctttggaggagcagtggtgggggtagctgtcacctccaggtatgattgagggaggaattgggtagaaactctccagacccatgcctccaatggcaggatgctgcctttcccacctgagaggggaccctgtccatgtgcagcctcatcagagcctcaccctgggaggatgccgtggcgtctcctcccaggagccagatcagtgcgagtgtgactgaaaatgcctcatcacttaagcaccaaagccagtgatcagcagctcttctgttcctgtgtcttctgtttttttctggtgaatcgttgcttgctgtggacttggtggaggactcagaggggaggaaaggctgggccccgagt

SEQ ID NO: 37 - DNA, 486 bp, Homo sapiens
accaaagagggcataataatcaagtctcacagtaggggttcaccatcctccaagtgaaaaacattgttccgaatgggctttccacaggctacacacacaaaacaggaaacatgccaagtttgtttcaacgcattgatgacttctccaaggatcttcctttggcatcgaccacattcaggggcaaagaatttctcatagcacagctcacaatacagggctcctttctcctctacaaatccaatgtaggccattgtatttttgcagtgagcgcagttgaattcttctgggtgccaagatttccccagtgccactaagaatggtcctctgatgacctggttacaatgggcgcacatcggagttcgtttccctgctggaatgtgctcagctctttgcactaaagtgtcctggtcacttggctgggtttgtcccaaagctgagttggctggtgccactgatcctgagtaagtagcgctgtttgagattctt

SEQ ID NO: 38 - DNA, 611 bp, Homo sapiens
  modified_base (455)..(579): N = A, C, G or T/U
acattcatttgaaagactaaaattaggtataagaacatgatttgaaaggatatttatagcctggatatacatgaaaaagtaagtaactatataaatgaagagtcataagtttacataaatataagaaacattaaattctaaaatattttctctatggtattcatgaatttttcactaattaaaaactctgtaataaaatatcttagggtccatataacaagtattaacaagattaaagattccactggattccaaaatgtagtctttgttggattcaaatgctgcataatcatctgtagaagtagtgtgggtaaacaacaggagactttagcaggagtaagtcctgactgtagcagaatccaacctctgtgttcctgatacattgccgtccatctgcatcttctttggctgcatagagggggaagacatataagaagtcacccggaagtttgcanggagagtctctgctgacccaacagctaagccacgaatgtcctttggtctaaactttttctccatgaaggtgctctcctaaagctttatcatcatcttcatcaaaccttcatcaatccccatgancaaaaggtgttaaattctcttccaagacacac

SEQ ID NO: 39 - DNA, 256 bp, Homo sapiens
actctttagagcagcagacagctgcagaatttgcagcaagtagcagacatctcagagatagcaaagtcctaaccccttgattagaaaaacagggagacagaagatttaaaacccactttttaaactgaggaagtctcaaataacttagcacatttatagtatcccctttacaatttttaaggctcattttggaaagtgtgtattaccataaacatggtctgcaacatgaggtgactgtaaggcaggttctgtttcc

SEQ ID NO: 40 - DNA, 403 bp, Homo sapiens
acatgaactcagggccggttgtggccatggtctgggaggggctgaacgtggtgaagacaggccgagtgatgcttggggagaccaatccagcagattcaaagccaggcaccattcgtggggacttctgcattcaggttggcaggaacatcattcatggcagtgattcagtaaaaagtactgaaaaagaaatcagcctatggtttaagcctgaagaactggttgactacaagtcttgtgctcatgactgggtctatgaataagaggtggacacaacagcagtctccttcagcacggcgtggtgtgtccctggacacagctcttcattccattgacttagaggcaacaggattgatcattcttttatagagcatatttgccaataaagcttttggaagccggaaaa

SEQ ID NO: 41 - DNA, 670 bp, Homo sapiens
gaagaatgtcagcccaggttcccatgaacatgaccatcacaggttgtatgatgacgttttacaggactacgccggctgtgctgttctggcagtggattaaccagtccttcaatgccgtcgtcaattacaccaacagaagtggagacgcacccctcactgtcaatgagttgggaacagcttacgtttctgcaacaactggtgccgtagcaacagctctaggactcaatgcattgaccaagcatgtctcaccactgataggacgttttgttccctttgctgccgtagctgctgctaattgcattaatattccattaatgaggcaaagggaactcaaagttggcattcccgtcacggatgagaatgggaaccgcttgggggagtcggcgaacgctgcgaaacaagccatcacgcaagttgtcgtgtccaggattctcatggcagcccctggcatggccatccctccattcattatgaacactttggaaaagaaagcctttttgaagaggttcccatggatgagtgcacccattcaagttgggttagttggcttctgtttggtgtttgctacacccctgtgttgtgccctgtttcctcagaaaagttccatgtctgtgacaagcttggaggccgagttgcaagctaagatccaagagagccatcctgaattgcg

SEQ ID NO: 42 - DNA, 386 bp, Homo sapiens
acaagaactttcggcccgacgaccccacccgcaaaaccaaagccctgctgcagatggtccagcagtttggggtggattttgagaagaggatcgagggctcaggagatcaggtggacactctggagctctccgggggcgcccgaatcaatcgcatcttccacgagcggttcccatttgagctggtgaagatggagtttgacgagaaggacttacgacgggagatcagctatgccattaagaacatccatggagtcaggaccgggcttttcaccccggacttggcattcgaggccattgtgaaaaagcaggtcgtcaagctgaaagagccctgtctgaaatgtgtcgacctggttatccaggagctaatcaatacagttaggcagtgt

SEQ ID NO: 43 - DNA, 547 bp, Homo sapiens
actacaactatgacaatgcttgacctacatttctaaaataaaaattcacattttttgataaataaactacagttttaccagaaattactatctaaatgtgtattagcagtattttttaaggtgaaattgccttggtatctaatgaatgtgtagacagggagataaaatgaaggattgccagactagttagaatagaatttaggattaggttagttttgaaaaatgatgttgtaatatatgggttctaacacatcctaccataaaaactggaggagatatgtgtaacctggttaatttgggatggtggacattttgggctaatactgacaaaatacatcttaggactagtatacatgtgacacggattgctaggaggaatgaaaaactaaactgtatagtttatattccgtaaaccattttataatgttcaaagattaggttttgttattgatagtattaaatacacagtttctcttaacagtgatgggtgaaaacattttaccggattatggaatgtttaccagaacatgttttgattcttgaatgt

SEQ ID NO: 44 - DNA, 640 bp, Homo sapiens
acctacaaacttaaacagaattccagactgcaccatatagatgtggccctcttattttgtgcacctgcaagttcactgtattacttcacgatttgatcagactaattctccttttttccactaagacaaatacctcactgtccaaactaaattgacccttcataaatgtatgtaaactttctgtgtttcaatatcctagcatgataaatttagctagttctctctaggcttgcctatcaatgggagctcttagatcttcttccttcttgatttaattaacaatgtaaatttgggctaaaagtcaattaattaattttgccagtttgaaaaactcgactactgatttgcttactaattcatctatttcctttatgttttttattttgctccctttctaacttctttagatatcaaattccattgatttttttcctttcttatgaataaagccatttttgttggaaaatgccaaaaaaaatttaaagggatccaattaacataaatttgattaacaacaaaataaatcaattgagtgaaaggaaacttaaccaaacttcaaaactatagaataataaatttagaattacttatccttaacatctacatttatagatagcatatatagcaataggagcatagt

SEQ ID NO: 45 - DNA, 435 bp, Homo sapiens
acacaagtcagtccaacagttagtgttaattactaataatatatgaaaaccctgccaacacaattgctgctacatcaccaatataattattaaccactgtcggaaaaacacacataaattcaggtaagactaaaagctgtctcacaaaaagaaaaaagaaatccaatggatccactaatgctatcaaaagggacatgcaggaatgtaacatgacatttttagaaatgtgtgtttctaaaaagaaaaaaaaatacactaaaatgccagtggactataattcattcaaaacatctttagtgttccttcccaaagatcttgatctgctcagtaattgcttcacaagatctatcacagccatcttttggagcgtatggttaggctggtcctcctgtggtggtaggggcagtctttttgaagctttaagtatctggtggt

SEQ ID NO: 46 - DNA, 291 bp, Homo sapiens
acctgttatgggagtatctgttgcattaggaacaattgaggaagtttgttcttttttccatcgatcaccacaactgcttttagaacttgacaacgtaatttctgttctttttcagaacagtaaagaaaggggtaaagaactgaaggaaatctgccattctcagtggacaggcaggcatgatgcttttgaaattttagtggaactcctgcaagcacttgttttatgtttagatggtataaatagtgacacaaatattagatggaataactatatagctggccgagcatttgt

SEQ ID NO: 47 - DNA, 491 bp, Homo sapiens
  modified_base (118, 309)..(448): N = A, C, G or T/U
acaaacctgcatggtgtttatgcacacagagatttgagaaccattgttctgaatgctgcttccatttgacaaagtgccgtgataatttttgaaaagagaagcaaacaatggtgtctcntttatgttcagcttataatgaaatctgtttgttgacttattaggactttgaattatttctttattaaccctctgagtttttgtatgtattattattaaagaaaaatgcaatcaggattttaaacatgtaaatacaaattttgtataacttttgatgacttcagtgaaattttcaggtagtctgagtaatanattgttttgccacttagaatagcatttgccacttagtattttaaaaaataattgttggagtatttattgtcagttttgttcacttgttatctaatacaaaattataaagccctcagagggttgggcacatctctttggnaaatagtttgcaacatatttaagagatacttgatgccaaaatg

SEQ ID NO: 48 - DNA, 682 bp, Homo sapiens
acctccaggaaatgtgctaggaataggaggccttcaagattttgtgctgaaatctgcaacactgtgtagcctgccatcctgcccaccatttataccactcaacttcgaagccactcctattgtgagagttgctgttgaaccaaaacatccaagtgaaatgcctcagctcgtaaaaggaatgaaactgttaaaccaggctgatccctgtgtccagattttaattcaggaaacgggagagcacgttttagtcacagcaggagaagtccaccttcagcgatgcctggatgacttaaaagaaaggtttgcaaagattcatatcagtgtatctgaacctattattccattcagagaaacaatcacaaaacccccaaaagttgacatggtcaatgaagaaataggcaaacagcaaaaagttgcagtcatacaccaaatgaaagaagatcaaagcaaaatccctgaaggaatccaagttgactctgacgggctaatcaccataacaactcccaataaacttgccacgctcagtgttcgagccatgccccttccagaagaagtcacccagattctggaagaaaatagtgatttgattcgttctatggagcagttgacatcctctttgaatgagggcgaaaatactcacatgattcatcagaagacccaagagaaaatttgggaattcaaa

SEQ ID NO: 49 - DNA, 532 bp, Homo sapiens
acaccaggcttgagtcagttttggccacatcccgccaaagctctccttccctgcgtggtttgaagggcctctagggttggggaacttgctgagacccacaggccaggtatatattcctcaagctgcttgcagggaggctcagagacagtaggaatgcctttcctgctcctggcctatcagtgcatattggccaggggaatttaatagaaccagagcaaatggaattgtattttaaactcactgggcagattccccaaagtgcttacatgtgtgggtgtaggggtggatttgaattccagctccaccactttttacctcaaagacttgcacgagctacctttctgcaccgccttctcatcagccctaaataaagaaactaacatgaagccctgcccaaggagagtctgagctcagactcgcctaaccctgcccccacctgatggtttgtctctacctgccctggtagctgaagaaaaaagacataatcttttggaagttctgtggtcctgtccattgcctaagaaacccgagt

SEQ ID NO: 50 - DNA, 692 bp, Homo sapiens
  modified_base (120): N = A, C, G or T/U
actataattcaatttctggcggtctcctgtctattggcaggaacaggcagcattgatccagcagccctcatgcaaatcggtgtcattccaacaaatgtccggcaacttatgtattatacntgaggcctcatcagcattcattgttgtgaagttaatgcctacaattgactcgccgattagtggatgtaatataacatcaatttcaagctataatgcaacagtgacaaaactcctacagccgatcggtgagaatttggagacaattaggaaccagttgattccaactcggaggagacgccgatttgcaggggtggtgattggattagctgcattaggagtagctactgccgcacaggtcactgccgcagtagcactagtaaaggcaaatgaaaatgctgcggctatactcaatctcaaaaatgcaatccaaaaaacaaatgcggcagttgcagacgtggtccaggccacacaatcactaggaacggcagttcaagcagttcaagatcatataaacagtgtggtaagtccagcaattacagcagccaactgtaaggcccaagatgctatcattggctcaatcctcaatctctatttgaccgagttgacaactatcttccacaatcaaattacaaaccctgcattgagtcctattacaattcaagctttaaggattctactggggagtacctcgg

SEQ ID NO: 51 - DNA, 685 bp, Homo sapiens
  modified_base (117, 126)..(134): N = A, C, G or T/U
acagcagcttctggcagcaagagaggttatagcatatatcttggaaagaaatgcctgcttattaccagcctattttgcagtcactgagatcaggaaactgtatcctgaaggcaaacnttccacacntggttacnttggaaacctagtatcagactttgtggataccttcaggcccacagcaaggataaactccatttgtggtcgctgtagtcttctgccagttgtaaataattcgggtgccatttgtaattcatggaaactggatcctgctactcttcgttttcctttgaaaggccttttgccatatgataaggatctgtttgaaccacagactgctttgttgagatatgtattggagcagccttattccagggatatggtctgcaatatgctaggtttaaataagcagcacaagcagcgctgccctgtgctggaggaccagttggtggatctggttgtttatgccatggagcgatctgagaccgaggagaagtttgacgatgggggaacaagccaactcctgtggcagcatctctcaagtcagctcattttctttgtgcttttccagtttgcaagttttccacatatggtgctttctcttcatcagaagttagcagggcgaggactgattaaaggcagagatcatcttatgtgggttctcctgcaattcatttctggaagtatt

SEQ ID NO: 52 - DNA, 44 bp, Artificial Sequence
  Description of Artificial Sequence  Synthetic Primer
ctaatacgactcactatagggctcgagcggccgcccgggcaggt

SEQ ID NO: 53 - DNA, 42 bp, Artificial Sequence
  Description of Artificial Sequence  Synthetic Primer
ctaatacgactcactatagggcagcgtggtcgcggccgaggt

SEQ ID NO: 54 - DNA, 22 bp, Artificial Sequence
  Description of Artificial Sequence  Synthetic Primer
ctaatacgactcactatagggc

SEQ ID NO: 55 - DNA, 22 bp, Artificial Sequence
  Description of Artificial Sequence  Synthetic Primer
tcgagcggccgcccgggcaggt

SEQ ID NO: 56 - DNA, 20 bp, Artificial Sequence
  Description of Artificial Sequence  Synthetic Primer
agcgtggtcgcggccgaggt

SEQ ID NO: 57 - protein, 17 aa, Artificial Sequence
  Description of Artificial Sequence  Synthetic Peptide
CGTYRKVVAARAPRKVL

(19) United States
(12) Patent Application Publication
(10) Pub. No.: US 2003/0100540 A1
(43) Pub. Date: May 29, 2003

(54) Identification of NSAID-regulated genes

(75) Inventors: Zhonghua Zhang, Nashville, TN (US); Raymond DuBois, Nashville, TN (US)

Correspondence Address: Steven L. Highlander, FULBRIGHT & JAWORSKI L.L.P., SUITE 2400, 600 CONGRESS AVENUE, AUSTIN, TX, 78701-3271, US

(73) Assignee: Vanderbilt University

(21) Appl. No.: 10/206,901

(22) Filed: Jul. 26, 2002

Related U.S. Application Data
(60) Provisional application No. 60/308,370, filed on Jul. 27, 2001.

(51) Int. Cl. A61K 31/60; A61K 31/415; C12Q 1/68; A61K 31/192

(52) U.S. Cl. 514/165; 435/6; 514/406; 514/570

(57) ABSTRACT

The invention is based on evidence that the anti-neoplastic effects of high dose NSAID treatments involve a complex pathway which involves alterations in the expression of genes which regulate a number of biological processes. The invention allows a more accurate molecular picture of cancer response to NSAIDS, and in so doing, allows one to assess the efficacy of a particular NSAID on a particular cancer, and to screen NSAIDS, and drugs generally, for their ability to produce NSAID-like anti-neoplastic effects.

The present application claims priority to co-pending U.S. Provisional Patent Application Serial No. 60/308,370 filed on Jul. 27, 2001. The entire text of the above-referenced disclosure is specifically incorporated herein by reference without disclaimer. The government owns rights in the present invention pursuant to grant numbers DK47297, P0 CA77839, P30 CA68485 from United States Public Health Services.

### BACKGROUND OF THE INVENTION

1. Field of the Invention

The present invention relates generally to the fields of molecular biology and cellular physiology. More particularly, it concerns the identification of gene involved in the response to NSAIDS.

2. Description of Related Art

Colorectal cancer (CRC) is the third-leading cancer diagnosed when including both genders, with about 130,000 Americans affected and 56,000 deaths from this disease each year (Landis et al., 1999). Several studies suggest that nonsteroidal anti-inflammatory drugs (NSAIDs), like aspirin and sulindac, have anti-neoplastic effects. Population-based studies consistently show a 40%-50% decrease in the relative risk of colorectal cancer in persons who are continuous users of NSAIDs (Thun et al., 1991). Studies of patients with familial adenomatous polyposis (FAP), an inherited predisposition for CRC, indicate that celecoxib, a selective COX-2 inhibitor, can reduce both the size and number of colorectal adenomas. Likewise, NSAIDs have been proven to inhibit tumorigenesis in mouse models of intestinal cancer (Jacoby et al., 2000; Oshima et al., 1996; Wechter et al., 1997), rat models of carcinogen induced colon cancer (DuBois et al., 1996; Rioux and Castonguay, 1998; Yoshimi et al., 1999) and xenografted human carcinomas in nude mice (Nishimura et al., 1999; Sawaoka et al., 1998; Sheng et al., 1997).

One of the most definitively characterized molecular targets of NSAIDs is the cyclooxygenase enzyme, which is hypothesized to be involved in the development of colorectal cancer (Oshima et al., 1996; Tsujii and DuBois, 1995). A large body of evidence suggests that inhibition of COX-2 and the resulting decrease in prostaglandin production may contribute to the anti-cancer effect of NSAIDs. Consistent with this effect, expression of COX-2 has been evaluated in different types of human malignancies and COX-2 levels are increased in intestinal adenomas and adenocarcinomas compared to matched normal mucosa (Eberhart et al., 1994; Williams et al., 1996). Expression of COX-2 in cultured cells confers a resistance to undergo programmed cell death (Chang et al., 2000; McGinty et al., 2000; Tsujii and DuBois, 1995), promotes angiogenesis (Tsujii et al., 1998; Williams et al., 2000a), stimulates metastatic potential (Tsujii et al., 1997) and modulates proliferation (Kinoshita et al., 1999). Inactivation of the COX-2 gene in mice is associated with decreased formation of intestinal adenomas (Oshima et al., 1996), with similar effects being seen in prostaglandin E receptor (EP₁) knockout mice (Watanabe et al., 1999). NS-398, a selective COX-2 inhibitor, is reported to suppress tumor growth in different types of cancer cell lines (Smith et al., 2000; Tsuji et al., 1996; Yoshimi et al., 1999). Yoshimi and Rioux et al. reported that NS-398 has chemopreventive effects in the AOM induced colon carcinogenesis rat model and NNK-induced lung cancer model (Rioux and Castonguay, 1998; Yoshimi et al., 1999). NS-398 was reported to induce apoptosis in human colon carcinoma cells (Hara et al., 1997), prostate carcinoma cells (Liu et al., 1998) and to have anti-angiogenic effects (Jones et al., 1999; Liu et al., 2000; Tsujii et al., 1998).

However, other investigators have indicated that COX-2 may not be the only target of NSAIDs. For example, recent evidence suggests that NSAIDs might regulate PPAR (He et al., 1999; Lehmann et al., 1997), NF-κB (Kopp and Ghosh, 1994; Yin et al., 1998), and the lipoxygenase pathway (Shureiqi et al., 2000) when given at high doses. Also at high concentrations, NSAIDs have anti-proliferative and pro-apoptotic effects on tumor cells that do not contain COX-2 (Hanif et al., 1996; Williams et al., 2000b; Zhang et al., 1999). It seems that both the COX-2 and non-COX-2 pathways can contribute to the anti-cancer effect of NSAIDs, which may involve the regulation of apoptosis, cell proliferation, angiogenesis and suppression of metastasis. These other molecular mechanisms by which NSAIDs exert their effects are not clear. Therefore, comparing the precise pattern of gene expression in cancer cells following NSAID treatment could provide important new information to help understand some of the underlying mechanisms responsible for their anti-neoplastic effect.

### SUMMARY OF THE INVENTION

In some embodiments, the invention relates to methods for predicting the response of a cancer cell to treatment with an NSAID comprising providing a cancer cell; contacting said cancer cell with an NSAID; and determining the NSAID's effect on the expression of a plurality of NSAID-modulated genes, wherein an effect similar to that seen in cancer cells that are responsive to NSAID treatment is predictive that said cancer cell also would be responsive to NSAID treatment.

For example, the cancer cell may be selected from the group consisting of a brain cancer cell, a head and neck cancer cell, an esophageal cancer cell, a lung cancer cell, a thyroid cancer cell, a stomach cancer cell, a colon cancer cell, a liver cancer cell, a kidney cancer cell, a prostate cancer cell, a breast cancer cell, a cervical cancer cell, an ovarian cancer cell, a testicular cancer cell, a rectal cancer cell, a skin cancer cell, and a blood cancer cell. Of course, any other form of cancer cell is contemplated within the scope of the invention.

In some preferred embodiments, the NSAID is a non-steroidal anti-inflammatory drug.

In some specific embodiments, the plurality of NSAID-modulated genes comprises at least one gene selected from the group consisting of the genes listed in Table 1 and Table 2. Of course, the plurality may comprise at least 2, 3, 4, 5, 6, 7, 8, 9, 10, 15, 20, 25, 30, 35, 40, 45, 50, or all 51 of the genes listed in Table 1 or 2, or any integer number between 1 and 51 of the genes listed in Table 1 and Table 2.

In other specific embodiments, the plurality of NSAID-modulated genes comprises one or more of the downregulated genes selected from the group consisting of SEQ. ID. NO: 1, SEQ. ID. NO: 2, SEQ. ID. NO: 3, SEQ. ID. NO: 6, SEQ. ID. NO: 7, SEQ. ID. NO: 18. In another specific embodiment, the plurality of NSAID-modulated genes comprises one or more of the upregulated genes selected from the group consisting of SEQ. ID. NO: 31, SEQ. ID. NO: 32, SEQ. ID. NO: 33, SEQ. ID. NO: 38, SEQ. ID. NO: 39, or SEQ. ID. NO: 42. In yet another specific embodiment, the plurality of NSAID-modulated genes comprises at least one of the downregulated genes selected from the group consisting of SEQ. ID. NO: 1, SEQ. ID. NO: 2, SEQ. ID. NO: 3, SEQ. ID. NO: 6, SEQ. ID. NO: 7, SEQ. ID. NO: 18, and at least one of the upregulated genes selected from the group consisting of SEQ. ID. NO: 31, SEQ. ID. NO: 32, SEQ. ID. NO: 33, SEQ. ID. NO: 38, SEQ. ID. NO: 39, or SEQ. ID. NO: 42. In still yet another specific embodiment, the plurality of NSAID-modulated genes comprises all of the downregulated genes selected from the group consisting of SEQ. ID. NO: 1, SEQ. ID. NO: 2, SEQ. ID. NO: 3, SEQ. ID. NO: 6, SEQ. ID. NO: 7, SEQ. ID. NO: 18. In still further specific embodiments, the plurality of NSAID-modulated genes comprises all of the upregulated genes selected from the group consisting of SEQ. ID. NO: 31, SEQ. ID. NO: 32, SEQ. ID. NO: 33, SEQ. ID. NO: 38, SEQ. ID. NO: 39, or SEQ. ID. NO: 42. In still another specific embodiment, the plurality of NSAID-modulated genes comprises all of the downregulated genes selected from the group consisting of SEQ. ID. NO: 1, SEQ. ID. NO: 2, SEQ. ID. NO: 3, SEQ. ID. NO: 6, SEQ. ID. NO: 7, SEQ. ID. NO: 18, and all of the upregulated genes selected from the group consisting of SEQ. ID. NO: 31, SEQ. ID. NO: 32, SEQ. ID. NO: 33, SEQ. ID. NO: 38, SEQ. ID. NO: 39, or SEQ. ID. NO: 42. In other specific embodiments, the plurality of NSAID-modulated genes comprises at least one gene selected from the group consisting of SEQ. ID. NO: 1, SEQ. ID. NO: 2, SEQ. ID. NO: 3, SEQ. ID. NO: 6, SEQ. ID. NO: 7, SEQ. ID. NO: 18, SEQ. ID. NO: 31, SEQ. ID. NO: 32, SEQ. ID. NO: 33, SEQ. ID. NO: 38, SEQ. ID. NO: 39, or SEQ. ID. NO: 42.

The plurality may comprise at least one gene that is not listed in Table 1 or Table 2. The plurality may further comprise 2, 3, 4, 5, 6, 7, 8, 9, 10, 15, 20, 25, 30, 35, 40, 45, 50, or more genes not listed in Table 1 or Table 2, or any integer number of genes not listed in Table 1 and Table 2. In some specific embodiments, the plurality comprises at least one gene listed in Table 5. Of course, more or all of the genes listed in Table 5 can be comprised in the plurality as well. Further, it is not necessary that any of the genes listed in Table 1, Table 2, and/or in Table 5 be comprised in the plurality. Rather, it is understood that those of skill in the art will likely be able to identify additional genes for use in the plurality, based upon the teachings of this specification.

In some preferred embodiments, determining comprises quantitating an mRNA for NSAID-modulated genes. The quantitating may comprise any method of quantitating including, for example, Northern analysis, densitometric scanning of a one- or two-dimensional gel, and/or nucleic acid amplification (for example by quantitative RT-PCR), and/or nucleic acid hybridization, quantitating NSAID-modulated polypeptides. Hybridization may comprise the use of a nucleic acid array, for example, a nucleic acid array disposed on a chip or wafer. Such a chip may comprise a glass or membrane that carries nucleic acid. Quantitating NSAID-modulated polypeptides may comprise, for example, densitometric scanning of a one- or two-dimensional gel and/or immunologic detection of said polypeptides. Immunologic detection may comprise Western blotting, ELISA, or RIA.

In some preferred embodiments, the cancer cell is obtained from a subject in need of cancer therapy or suspected of having cancer. In some cases, the method will comprise treating said subject.

The methods may further comprise treating a cancer cell, known to be responsive to said NSAID, with said NSAID, and comparing the expression of the same plurality of NSAID-modulated genes with the results obtained by determining the NSAID's effect on the expression of a plurality of NSAID-modulated genes.

In other embodiments, the invention relates to methods of predicting the efficacy of an NSAID in cancer therapy comprising providing a cancer cell; contacting said cancer cell with an NSAID; and determining the NSAID's effect on the expression of a plurality of NSAID-modulated genes, wherein an effect similar to that seen in cancer cells treated with known cancer-therapeutic NSAIDs is predictive that said NSAID also would be efficacious in cancer therapy. The cancer cells, NSAIDs, pluralities of NSAID-modulated genes, methods for determining NSAID effect, source of cancer cell, treatment, etc., are all as described above.

The invention also relates to methods for treating a subject with cancer comprising administering to said subject a composition that modulates the levels of one or more of NSAID-modulated polypeptides. In some specific embodiments, the composition increases the level of one or more of NSAID-stimulated polypeptides. Such NSAID-stimulated polypeptides may be translation products of the NSAID s-stimulated genes. The composition may decrease the level of one or more of NSAID-inhibited polypeptides, with such NSAID-inhibited polypeptides being, in some cases, translation products of the NSAID-inhibited genes.

These methods of treatment may further comprise administering to said subject two compositions, wherein a first composition increases the level of one or more of NSAID-stimulated polypeptides, and a second composition decreases the level of one or more of NSAID-inhibited polypeptides.

The cancer cell in these treatment embodiments may be any cancer, as described above.

The composition, in some embodiments, comprises an expression cassette comprising a nucleic acid encoding one of said NSAID-modulated polypeptides, and a promoter active cells of said subject. For example, said nucleic acid may encode an NSAID-stimulated polypeptide and be positioned sense to said promoter or encode an NSAID-inhibited polypeptides and be positioned anti-sense to said promoter. The expression cassette, in some embodiments, is incorporated in non-viral expression vector. The expression cassette may also be incorporated in viral expression vector, for example, an adenoviral expression vector, a herpesviral expression vector, a retroviral expression vector, a vaccinia viral expression vector, an adeno-associated viral expression vector or a polyoma viral expression vector. The promoter may be, for example, a constitutive promoter, a tumor specific promoter or an inducible promoter. In some cases, the expression cassette further comprises a polyadenylation signal.

In some specific embodiments, the composition is an expression cassette comprising a nucleic acid encoding ribozyme that cleaves the mRNA encoding one of said NSAID-inhibited polypeptides, and a promoter active cells of said subject. Also, the composition can be an expression cassette comprising a nucleic acid encoding single chain antibody that binds one of said NSAID-inhibited polypeptides, and a promoter active cells of said subject.

In some embodiments, the composition is a small molecule.

The methods of treatment may further comprise treating said subject with a chemotherapy, a radiotherapy, an immunotherapy or surgery. Additionally, these methods may further comprise monitoring the level of one or more NSAID-modulated polypeptides or transcript.

The invention also relates to methods of screening compounds for NSAID-like activity comprising: providing a cell; contacting said cell with a candidate compound; and determining the NSAID's effect on the expression of a plurality of NSAID-modulated genes, wherein an effect similar to that seen in cells treated with known NSAIDs is predictive that said candidate compound exhibits NSAID-like activity. In some preferred embodiments, the plurality of NSAID-modulated genes will be as described above. These methods may further comprise determining the expression of a plurality of NSAID-modulated genes in the absence of said candidate compound. Additionally, these methods may further comprise determining the expression of a plurality of NSAID-modulated genes in a similar cell following treatment with a known NSAID. In some preferred embodiments, the cell is a cancer cell. In these methods, determining may comprise quantitating an mRNA for NSAID-modulated genes, by, for example, nucleic acid amplification, nucleic acid hybridization (for example, using a nucleic acid array disposed on a chip or wafer), and/or quantitating NSAID-modulated polypeptides.

Some embodiments of the invention relate to a nucleic acid array comprising a plurality of nucleic acid segments comprising at least 15 consecutive bases of a nucleic acid encoding a plurality of NSAID-modulated polypeptides. The nucleic acid array may be disposed on a chip or wafer. The plurality of NSAID-modulated polypeptides, will, in some preferred embodiments, be as described above.

In other embodiments, the invention relates to a kit comprising a chip comprising a nucleic acid array comprising a plurality of nucleic acid segments comprising at least 15 consecutive bases of a nucleic acid encoding a plurality of NSAID-modulated polypeptides, the array being as described above.

### DESCRIPTION OF ILLUSTRATIVE EMBODIMENTS

Colorectal cancer causes 56,000 deaths each year in the U.S. (Landis et al., 1999). Nonsteroidal anti-inflammatory drugs (NSAIDs), such as aspirin, sulindac and celecoxib, have anti-neoplastic effects, either alone or in combination with other drugs. One of the most definitively characterized molecular targets of NSAIDs is the cyclooxygenase enzyme, which is hypothesized to be involved in the development of colorectal cancer (Oshima et al., 1996; Tsujii and DuBois, 1995). A large body of evidence suggests that inhibition of COX-2 and the resulting decrease in prostaglandin production may contribute to the anti-cancer effect of NSAIDs by blocking (a) COX-2 resistance to programmed cell death, (b) COX-2 promoted angiogenesis, (c) COX-2 stimulation of metastatic potential and (d) COX-2 modulation of proliferation. The precise mechanism(s) through which NSAIDs act is, however, still not known.

Many different techniques have been developed to obtain an inventory of differential transcripts between two populations of mRNAs. Identification and isolation of differentially expressed transcripts is generally achieved by one of the following methods: differential display (DD) and related techniques (Liang and Pardee, 1992); representational difference analysis (RDA) (Lisitsyn et al., 1993); enzymatic degradation subtraction (Zeng et al., 1994); techniques involving physical removal of common sequences (Akopian and Wood, 1995; Deleersnijder et al., 1996); suppression subtractive hybridization; differential screening or cDNA micro-array analysis (Drmanac and Drmanac, 1999). Despite the fact that all these methods have proven successful in the isolation of differentially expressed genes, they all possess some intrinsic drawbacks.

Two other approaches—suppression subtractive hybridization (SSH) (Diatchenko et al., 1996) and differential screening (DS) (Maser and Calvet, 1995)—are powerful tools used to identify and isolate cDNAs of differentially expressed genes (Kuang et al., 1998; von Stein et al., 1997; Zuber et al., 2000). SSH is a PCR-based subtraction method which selectively amplifies target cDNA fragments and simultaneously suppresses amplification of non-target cDNAs. DS is a simple method based on reverse Northern blot analysis. These cloning techniques have the advantage over high density DNA micro-array analysis in that they can detect previously unidentified genes.

In particular, the strength of SSH stems from a process called normalization, which equalizes the wide differences in abundance of different mRNA species. This allows for the recovery of abundantly expressed transcripts (i.e., those encoding cytoskeletal proteins), and of low-copy-number mRNAs (i.e., those encoding transcription factors). Compared to other methods which restrict the analysis only to differences at the 3′-end of cDNAs or require multiple rounds of subtraction, SSH is simple and covers all the portions of the cDNA sequence. Beside this, SSH also results in the isolation of relatively long cDNA sequences, sufficient to permit database searching for homology to common motifs of predicted translation products.

The present inventors employed both SSH and DS to examine the expression profiles of cells treated with NSAIDs. The results reported here provide evidence that the anti-neoplastic effects of high dose NSAID treatments involve a complex pathway which involves alterations in the expression of genes which regulate a number of biological processes. From these studies, the present inventors can now provide a more accurate molecular picture of cancer response to NSAIDs, and in so doing, allow one to assess the efficacy of a particular NSAID on a particular cancer, and to screen NSAIDs, and drugs generally, for their ability to produce NSAID-like anti-neoplastic effects.

### I. NSAIDs

NSAIDs are anti-inflammatory agents that are not steroids. In addition to anti-inflammatory actions, they have analgesic, antipyretic, and platelet-inhibitory actions. They are used primarily in the treatment of chronic arthritic conditions and certain soft tissue disorders associated with pain and inflammation. They act by blocking the synthesis of prostaglandins by inhibiting cyclooxygenase, which converts arachidonic acid to cyclic endoperoxides, precursors of prostaglandins. Inhibition of prostaglandin synthesis accounts for their analgesic, antipyretic, and platelet-inhibitory actions; other mechanisms may contribute to their anti-inflammatory effects. Certain NSAIDs also may inhibit lipoxygenase enzymes or phospholipase C or may modulate T-cell function.

### 1. Sulindac and its Major Metabolites, Sulindac Sulfone and Sulindac Sulfide

Sulindac is a non-steroidal, anti-inflammatory indene derivative with the following chemical designation; (Z)-5-Fluoro-2-methyl-1-((4(methylsulfinyl)phenyl)methylene) 1H-indene-3-acetic acid (Physician's Desk Reference, Medical Economics Data, Montville, N.J., 1745-1747, 1999). The sulfinyl moiety is converted in vivo by reversible reduction to the sulfide metabolite and by irreversible oxidation to the sulfone metabolite. Available evidence indicates that the sulfide derivative is the biologically active compound. Based on this, sulindac is defined as a prodrug, and appears to be inactive or relatively weak in many tests where little or no metabolism can occur. Sulindac (Clinoril®) is available as 150- and 200-mg tablets. The most common dosage for adults is 150 to 200 mg twice a day, with a maximal daily dose of 400 mg. After oral administration, about 90% of the drug is absorbed. Peak plasma levels are achieved in about 2 hours in fasting patients and 3 to 4 hours when administered with food. The mean half-life of sulindac is 7.8 hours: the mean half-life of the sulfide metabolite is 16.4 hours. U.S. Pat. Nos. 3,647,858 and 3,654,349 cover preparations of sulindac.

Sulindac is currently indicated for the acute and long-term relief of signs and symptoms of osteoarthritis, rheumatoid arthritis, ankylosing spondylitis, acute gout, and acute painful shoulder. The analgesic and anti-inflammatory effects exerted by sulindac (400 mg per day) are comparable to those achieved by aspirin (4 g per day), ibuprofen (1200 mg per day), indomethacin (125 mg per day), and phenylbutazone (400 to 600 mg per day). Side effects of sulindac include mild gastrointestinal effects in nearly 20% of patients, with abdominal pain and nausea being the most frequent complaints. CNS side effects are seen in up to 10% of patients, with drowsiness, headache, and nervousness being those most frequently reported. Skin rash and pruritus occur in 5% of patients. Chronic treatment with sulindac can lead to serious gastrointestinal toxicity such as bleeding, ulceration, and perforation.

The potential use of sulindac for chemoprevention of cancers, and in particular colorectal polyps, has been well studied. Three U.S. Pat. Nos. 5,814,625, 5,843,929, and 6,258,845 detail potential chemopreventive uses of sulindac in humans. Doses of sulindac claimed in U.S. Pat. No. 5,814,625 range from 10 mg to 1500 mg per day, with preferred doses of 50 mg to 500 mg per day. However, at the higher doses, the biggest problem with the use of sulindac as a single agent in chemoprevention is its well-known toxicities and moderately high risk of intolerance. The elderly appear to be especially vulnerable, as the incidence of side effects is higher in those over the age of 60. It is noted that this age group is most likely to develop colorectal cancer, and therefore, most likely to benefit from chemoprevention.

### 2. Piroxicam

A non-steroidal anti-inflammatory agent that is well established in the treatment of rheumatoid arthritis and osteoarthritis with the following chemical designation; 4-Hydroxy-2-methyl-N-2-pyridyl-2H-1,2-benzothiazine-3-carboxamide 1,1-dioxide. Its usefulness also has been demonstrated in the treatment of musculoskeletal disorders, dysmenorrhea, and postoperative pain. Its long half-life enables it to be administered once daily. The drug has been shown to be effective if administered rectally. Gastrointestinal complaints are the most frequently reported side effects.

### 3. Aspirin

The prototypical analgesic used in the treatment of mild to moderate pain. It has anti-inflammatory and antipyretic properties and acts as an inhibitor of cyclooxygenase which results in the inhibition of the biosynthesis of prostaglandins. Aspirin also inhibits platelet aggregation and is used in the prevention of arterial and venous thrombosis. (From Martindale, The Extra Pharmacopoeia, 30th ed, p5)

### 4. Indomethacin

A non-steroidal anti-inflammatory agent that is used in the treatment of osteoarthritis, acute gouty arthritis, rheumatoid arthritis, and ankylosing spondylitis. Due to its action as a prostaglandin synthase inhibitor, indomethacin has also been used to delay premature labor.

### 5. Celecoxib and Other Selective COX-2 Inhibitors

One of the newest NSAIDs is celecoxib. Sold by Searle under the trade name CELEBREX™, celecoxib is chemically designated as 4-[5-(4-methyphenyl)-3-(trifluoromethyl)-1H-pyrazol-1-yl] benzenesulfonamide. The empirical formula is C₁₇H₁₄F₃N₃O₂S, and the molecular weight is 381.38. CELEBREX™ and VIOXX™ is marketed in 100 or 200 mg oral capsules.

Celecoxib exhibits anti-inflammatory, analgesic and antipyreti activities in animal models. The mechanism of action is thought to be the result of inhibition of prostaglandin synthesis. The enzyme cyclooxygenase-2, or “COX-2,” is an important enzyme in this pathway. Selective inhibition of COX-2 (the related enzyme COX-1 is not inhibited) is a unique characteristic of celecoxib, and is believed to reduce potential gastrointestinal toxicities associated with inhibition of COX-1.

The inventors have also evaluated the effect of other selective COX-2 inhibitors, such as NS-398, and have found that they have similar effects to those observed using Celecoxib.

### 6. Combination Therapy

In order to improve a cancer therapy, it is often common to combine two or more therapeutic modalities. Thus, the present invention envisions the combination of multiple NSAIDs following their identification as being useful for treating cancers generally, or a particular cancer. In addition, using lower doses of two or more NSAIDs may make it possible to reduce the side effects or toxicities associated with higher doses of individual NSAIDs.

It also may be desirable to combine and NSAID therapy with another type of anti-cancer treatment (see U.S. Pat. No. 6,258,845). Generally, an “anti-cancer” agent is any agent capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer. More generally, these other compositions would be provided in a combined amount effective to kill or inhibit proliferation of the cell. This may be achieved by contacting the cell with a single composition or pharmacological formulation that includes both agents, or by contacting the cell with two distinct compositions or formulations, at the same time, wherein one composition includes the NSAID and the other includes the second agent(s).

Alternatively, the NSAID therapy may precede or follow the other agent treatment by intervals ranging from minutes to weeks. In embodiments where the other agent and NSAID are applied separately to the cell, one would generally ensure that a significant period of time did not expire between the time of each delivery, such that the agent and expression construct would still be able to exert an advantageously combined effect on the cell. In such instances, it is contemplated that one may contact the cell with both modalities within about 12-24 h of each other and, more preferably, within about 6-12 h of each other. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

Various combinations may be employed, where the NSAID is “A” and the secondary agent, such as radio- or chemotherapy, is “B”:

A/B/A B/A/B B/B/A A/A/B A/B/B B/A/A A/B/B/B B/A/B/B

B/B/B/A B/B/A/B A/A/B/B A/B/A/B A/B/B/A B/B/A/A

B/A/B/A B/A/A/B A/A/A/B B/A/A/A A/B/A/A A/A/B/A

Administration of an NSAID to a patient will follow general protocols for the administration of chemotherapeutics, taking into account the toxicity, if any, for the particular patient. It is expected that the treatment cycles would be repeated as necessary. It also is contemplated that various standard therapies, as well as surgical intervention, may be applied in combination with the described hyperproliferative cell therapy.

### a. Chemotherapy

Cancer therapies also include a variety of combination therapies with both chemical and radiation based treatments. Combination chemotherapies include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, famesyl-protein tansferase inhibitors, transplatinum, 5-fluorouracil, vincristin, vinblastin and methotrexate, or any analog or derivative variant of the foregoing.

### b. Radiotherapy

Other factors that cause DNA damage and have been used extensively include what are commonly known as γ-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing or stasis, both agents are delivered to a cell in a combined amount effective to kill the cell or prevent it from dividing.

### c. Immunotherapy

Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

Immunotherapy, thus, could be used as part of a combined therapy, in conjunction with NSAID therapy. The general approach for combined therapy is discussed below. Generally, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155.

### II. GENES IDENTIFIED AS RELEVANT TO NSAID RESPONSE

Table 1 contains an exemplary list of genes that are down-regulated by treatment with NSAIDs. Table 2 contains an exemplary list of genes that are up-regulated by treatment with NSAIDs. Those of skill in the art will be able to use some or all of these exemplary gene sequences in the context of the invention, as well as other gene sequences that are not listed in Table 1 or Table 2 but that are affected by treatment with NSAIDs.

TABLE 1
List of Down-regulated genes by treatment of NASIDs (25)

| CLONE | | | SEQ ID |
|---|---|---|---|
| NO | GENE | Function | NO |
| D101 | restricted expressed | Cell proliferation marker | 1 |
| | proliferation assoc- | | |
| | iated protein 100 | | |
| D104 | KIAA0101 | Unknown | 2 |
| D108 | cyclin K | a member of the cyclin | 3 |
| | | superfamily | |
| D113 | KIAA0121 | Unknown | 4 |
| D212 | glutathione | a kind of peroxidase | 5 |
| | peroxidase 1 | | |
| D213 | prothymosin alpha | Involved in cell proliferation and | 6 |
| | | regulated by oncogene c-myc | |
| D217 | Rad50 | DNA recombinational repair | 7 |
| | | gene | |
| D222 | KIAA0974 | Unknown | 8 |
| D224 | MSSP-2 | c-myc single-strand binding | 9 |
| | | protein | |
| D225 | H2A histone family, | a member of histone family | 10 |
| | member Z (H2AFZ) | | |
| D227 | RA-regulated | Unclear | 11 |
| | nuclear matrix- | | |
| | associated protein | | |
| D235 | hRlf beta subunit | Transcription factor | 12 |
| | (p102 protein) | | |
| D236 | signal recognition | Protein targeting to the ER | 13 |
| | particle 72kD | memberane | |
| D238 | Homo sapiens endo- | Involved in protein C | 14 |
| | thelial protein C | anticogulant pathway | |
| | receptor (PROCR) | | |
| D239 | F-box only protein 5 | A protein that has a F-box | 15 |
| | (FBXO5) | domain which is involved in | |
| | | protein interaction | |
| D240 | New | Unknown | 16 |
| D241 | New | Unknown | 17 |
| D242 | Human tissue factor | Initiation of coagulation and also | 18 |
| | | highly expressed in some tumors | |
| D307 | neuromedin U | an endogenous ligand for an | 19 |
| | (NMU) | orphan receptor GPR66 | |
| D315 | New | Unknown | 20 |
| D316 | transcription factor | Transcription factor | 21 |
| | Dp-1 (TFDP1) | | |
| D320 | New | Unknown | 22 |
| D323 | New | Unknown | 23 |
| D325 | New | Unknown | 24 |
| D328 | KIAA1266 | Unknown | 25 |

TABLE 2
List of Up-regulated genes by treatment of NASIDs (26)

| CLONE | | | SEQ ID |
|---|---|---|---|
| NO | GENE | Function | NO |
| N107 | KIAA1252 | Unknown | 26 |
| N110 | New | Unknown | 27 |
| N112 | six transmembrane epithelial | Tumor membrane | 28 |
| | antigen of the Prostate (STEAP) | antigen | |
| N116 | mRNA for PCDH7 (BH-Pcdh) | A member of the | 29 |
| | | cadherin family | |
| N117 | New | Unknown | 30 |
| N118 | Human pdcd4 gene | Apoptosis related | 31 |
| | | gene | |
| N122 | Human FAT gene | A member of the | 32 |
| | | cadherin family | |
| N123 | IMP (inosine monophosphate) | Regulation of cell | 33 |
| | dehydrogenase 2 | growth and | |
| | | differentiation | |
| N127 | New | Unknown | 34 |
| N130 | New | Unknown | 35 |
| N135 | New | Unknown | 36 |
| N207 | LIM protein (similar to rat pro- | Involved in protein | 37 |
| | tein kinase C-binding enigma) | interaction | |
| | (LIM) | | |
| N208 | LAR protein tyrosine | Involved in LAR | 38 |
| | phosphatase | signaling | |
| N211 | Human long-chain acyl-CoA | Long-chain acyl- | 39 |
| | synthetase | CoA synthesis | |
| N215 | c-myc transcription factor | Suppressor of tumor | 40 |
| | | metastasis | |
| N218 | New | Unknown | 41 |
| N221 | dynamin 2 (DNM2) | Apoptosis related | 42 |
| N222 | New | Unknown | 43 |
| N223 | Homologous to KIAA1051 | Unknown | 44 |
| N228 | New | Unknown | 45 |
| N231 | Interferon-induced protein | A latent protein | 46 |
| | kinase PKR | kinas | |
| N234 | Proteinase activated receptor-2 | Unclear | 47 |
| N302 | New | Unknown | 48 |
| N327 | New | Unknown | 49 |
| N339 | New | Unknown | 50 |
| N342 | Human vitamin D3 receptor | Nuclear receptor | 51 |
| | interacting protein | cofactor | |

### III. METHODS OF ASSAYING FOR ALTERATIONS IN GENE EXPRESSION

Thus, in accordance with the present invention, methods are provided for the assaying of gene expression in response to NSAID treatment. As discussed above, the principle applications of this assay are to: (a) determine if a given cancer cell will respond to one or more NSAIDs; (b) to determine if a given NSAID will be effective against one or more cancers; and (c) to screen compounds in general for activity that mimics or parallels NSAID anti-neoplastic effects. In each of these assays, the expression of a particular set of genes, set forth in the preceding section, will be measured. The following is a discussion of various aspects of the method.

### 1. Hybridization

There are a variety of ways by which one can assess gene expression. These methods either look at protein or at mRNA levels. Methods looking at mRNAs all fundamentally rely, at a basic level, on nucleic acid hybridization. Hybridization is defined as the ability of a nucleic acid to selectively form duplex molecules with complementary stretches of DNAs and/or RNAs. Depending on the application envisioned, one would employ varying conditions of hybridization to achieve varying degrees of selectivity of the probe or primers for the target sequence.

Typically, a probe or primer of between 13 and 100 nucleotides, preferably between 17 and 100 nucleotides in length up to 1-2 kilobases or more in length will allow the formation of a duplex molecule that is both stable and selective. Molecules having complementary sequences over contiguous stretches greater than 20 bases in length are generally preferred, to increase stability and selectivity of the hybrid molecules obtained. One will generally prefer to design nucleic acid molecules for hybridization having one or more complementary sequences of 20 to 30 nucleotides, or even longer where desired. Such fragments may be readily prepared, for example, by directly synthesizing the fragment by chemical means or by introducing selected sequences into recombinant vectors for recombinant production.

For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting specific mRNA transcripts. It is generally appreciated that conditions can be rendered more stringent by the addition of increasing amounts of formamide.

For certain applications, for example, lower stringency conditions may be used. Under these conditions, hybridization may occur even though the sequences of the hybridizing strands are not perfectly complementary, but are mismatched at one or more positions. Conditions may be rendered less stringent by increasing salt concentration and/or decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Hybridization conditions can be readily manipulated depending on the desired results.

In other embodiments, hybridization may be achieved under conditions of, for example, 50 mM Tris-HCl (pH 8.3), 75 mM KCl, 3 mM MgCl₂, 1.0 mM dithiothreitol, at temperatures between approximately 20° C. to about 37° C. Other hybridization conditions utilized could include approximately 10 mM Tris-HCl (pH 8.3), 50 mM KCl, 1.5 mM MgCl₂, at temperatures ranging from approximately 40° C. to about 72° C.

In certain embodiments, it will be advantageous to employ nucleic acids of defined sequences of the present invention in combination with an appropriate means, such as a label, for determining hybridization. A wide variety of appropriate indicator means are known in the art, including fluorescent, radioactive, enzymatic or other ligands, such as avidin/biotin, which are capable of being detected. In preferred embodiments, one may desire to employ a fluorescent label or an enzyme tag such as urease, alkaline phosphatase or peroxidase, instead of radioactive or other environmentally undesirable reagents. In the case of enzyme tags, colorimetric indicator substrates are known that can be employed to provide a detection means that is visibly or spectrophotometrically detectable, to identify specific hybridization with complementary nucleic acid containing samples.

In general, it is envisioned that the probes or primers described herein will be useful as reagents in solution hybridization, as in PCR™, for detection of expression of corresponding genes, as well as in embodiments employing a solid phase. In embodiments involving a solid phase, the test DNA (or RNA) is adsorbed or otherwise affixed to a selected matrix or surface. This fixed, single-stranded nucleic acid is then subjected to hybridization with selected probes under desired conditions. The conditions selected will depend on the particular circumstances (depending, for example, on the G+C content, type of target nucleic acid, source of nucleic acid, size of hybridization probe, etc.). Optimization of hybridization conditions for the particular application of interest is well known to those of skill in the art. After washing of the hybridized molecules to remove non-specifically bound probe molecules, hybridization is detected, and/or quantified, by determining the amount of bound label. Representative solid phase hybridization methods are disclosed in U.S. Pat. Nos. 5,843,663, 5,900,481 and 5,919,626. Other methods of hybridization that may be used in the practice of the present invention are disclosed in U.S. Pat. Nos. 5,849,481, 5,849,486 and 5,851,772. The relevant portions of these and other references identified in this section of the Specification are incorporated herein by reference.

### 2. Amplification of Nucleic Acids

Since many nucleic acids, especially mRNAs, are in low abundance, nucleic acid amplification greatly enhances the ability to assess expression. The general concept is that nucleic acids can be amplified using paired primers flanking the region of interest. The term “primer,” as used herein, is meant to encompass any nucleic acid that is capable of priming the synthesis of a nascent nucleic acid in a template-dependent process. Typically, primers are oligonucleotides from ten to twenty and/or thirty base pairs in length, but longer sequences can be employed. Primers may be provided in double-stranded and/or single-stranded form, although the single-stranded form is preferred.

Pairs of primers designed to selectively hybridize to nucleic acids corresponding to selected genes are contacted with the template nucleic acid under conditions that permit selective hybridization. Depending upon the desired application, high stringency hybridization conditions may be selected that will only allow hybridization to sequences that are completely complementary to the primers. In other embodiments, hybridization may occur under reduced stringency to allow for amplification of nucleic acids contain one or more mismatches with the primer sequences. Once hybridized, the template-primer complex is contacted with one or more enzymes that facilitate template-dependent nucleic acid synthesis. Multiple rounds of amplification, also referred to as “cycles,” are conducted until a sufficient amount of amplification product is produced.

The amplification product may be detected or quantified. In certain applications, the detection may be performed by visual means. Alternatively, the detection may involve indirect identification of the product via chemilluminescence, radioactive scintigraphy of incorporated radiolabel or fluorescent label or even via a system using electrical and/or thermal impulse signals.

A number of template dependent processes are available to amplify the oligonucleotide sequences present in a given template sample. One of the best known amplification methods is the polymerase chain reaction (referred to as PCR™) which is described in detail in U.S. Pat. Nos. 4,683,195, 4,683,202 and 4,800,159, and in Innis et al., 1988, each of which is incorporated herein by reference in their entirety.

A reverse transcriptase PCR™ amplification procedure may be performed to quantify the amount of mRNA amplified. Methods of reverse transcribing RNA into cDNA are well known (see Sambrook et al., 2000). Alternative methods for reverse transcription utilize thermostable DNA polymerases. These methods are described in WO 90/07641. Polymerase chain reaction methodologies are well known in the art. Representative methods of RT-PCR are described in U.S. Pat. No. 5,882,864.

Another method for amplification is ligase chain reaction (“LCR”), disclosed in European Application No. 320 308, incorporated herein by reference in its entirety. U.S. Pat. No. 4,883,750 describes a method similar to LCR for binding probe pairs to a target sequence. A method based on PCR™ and oligonucleotide ligase assay (OLA), disclosed in U.S. Pat. No. 5,912,148, may also be used.

Alternative methods for amplification of target nucleic acid sequences that may be used in the practice of the present invention are disclosed in U.S. Pat. Nos. 5,843,650, 5,846,709, 5,846,783, 5,849,546, 5,849,497, 5,849,547, 5,858,652, 5,866,366, 5,916,776, 5,922,574, 5,928,905, 5,928,906, 5,932,451, 5,935,825, 5,939,291 and 5,942,391, GB Application No. 2 202 328, and in PCT Application No. PCT/US89/01025, each of which is incorporated herein by reference in its entirety.

Qbeta Replicase, described in PCT Application No. PCT/US87/00880, may also be used as an amplification method in the present invention. In this method, a replicative sequence of RNA that has a region complementary to that of a target is added to a sample in the presence of an RNA polymerase. The polymerase will copy the replicative sequence which may then be detected.

An isothermal amplification method, in which restriction endonucleases and ligases are used to achieve the amplification of target molecules that contain nucleotide 5′-[α-thio]-triphosphates in one strand of a restriction site may also be useful in the amplification of nucleic acids in the present invention (Walker et al., 1992). Strand Displacement Amplification (SDA), disclosed in U.S. Pat. No. 5,916,779, is another method of carrying out isothermal amplification of nucleic acids which involves multiple rounds of strand displacement and synthesis, i.e., nick translation.

Other nucleic acid amplification procedures include transcription-based amplification systems (TAS), including nucleic acid sequence based amplification (NASBA) and 3SR (Kwoh et al., 1989; Gingeras et al., PCT Application WO 88/10315, incorporated herein by reference in their entirety). European Application No. 329822 disclose a nucleic acid amplification process involving cyclically synthesizing single-stranded RNA (“ssRNA”), ssDNA, and double-stranded DNA (dsDNA), which may be used in accordance with the present invention.

PCT Application WO 89/06700 (incorporated herein by reference in its entirety) disclose a nucleic acid sequence amplification scheme based on the hybridization of a promoter region/primer sequence to a target single-stranded DNA (“ssDNA”) followed by transcription of many RNA copies of the sequence. This scheme is not cyclic, i.e., new templates are not produced from the resultant RNA transcripts. Other amplification methods include “race” and “one-sided PCR” (Frohman, 1990; Ohara et al., 1989).

### 3. Detection of Nucleic Acids

Following any amplification, it may be desirable to separate the amplification product from the template and/or the excess primer. In one embodiment, amplification products are separated by agarose, agarose-acrylamide or polyacrylamide gel electrophoresis using standard methods (Sambrook et al., 2000). Separated amplification products may be cut out and eluted from the gel for further manipulation. Using low melting point agarose gels, the separated band may be removed by heating the gel, followed by extraction of the nucleic acid.

Separation of nucleic acids may also be effected by chromatographic techniques known in art. There are many kinds of chromatography which may be used in the practice of the present invention, including adsorption, partition, ion-exchange, hydroxylapatite, molecular sieve, reverse-phase, column, paper, thin-layer, and gas chromatography as well as HPLC.

In certain embodiments, the amplification products are visualized. A typical visualization method involves staining of a gel with ethidium bromide and visualization of bands under UV light. Alternatively, if the amplification products are integrally labeled with radio- or fluorometrically-labeled nucleotides, the separated amplification products can be exposed to x-ray film or visualized under the appropriate excitatory spectra.

In one embodiment, following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, or another binding partner carrying a detectable moiety.

In particular embodiments, detection is by Southern blotting and hybridization with a labeled probe. The techniques involved in Southern blotting are well known to those of skill in the art (see Sambrook et al, 2000). One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

Other methods of nucleic acid detection that may be used in the practice of the instant invention are disclosed in U.S. Pat. Nos. 5,840,873, 5,843,640, 5,843,651, 5,846,708, 5,846,717, 5,846,726, 5,846,729, 5,849,487, 5,853,990, 5,853,992, 5,853,993, 5,856,092, 5,861,244, 5,863,732, 5,863,753, 5,866,331, 5,905,024, 5,910,407, 5,912,124, 5,912,145, 5,919,630, 5,925,517, 5,928,862, 5,928,869, 5,929,227, 5,932,413 and 5,935,791, each of which is incorporated herein by reference.

### 4. Nucleic Acid Arrays

Microarrays comprise a plurality of polymeric molecules spatially distributed over, and stably associated with, the surface of a substantially planar substrate, e.g., biochips. Microarrays of polynucleotides have been developed and find use in a variety of applications, such as screening and DNA sequencing. One area in particular in which microarrays find use is in gene expression analysis.

In gene expression analysis with microarrays, an array of “probe” oligonucleotides is contacted with a nucleic acid sample of interest, i.e., target, such as polyA mRNA from a particular tissue type. Contact is carried out under hybridization conditions and unbound nucleic acid is then removed. The resultant pattern of hybridized nucleic acid provides information regarding the genetic profile of the sample tested. Methodologies of gene expression analysis on microarrays are capable of providing both qualitative and quantitative information.

A variety of different arrays which may be used are known in the art. The probe molecules of the arrays which are capable of sequence specific hybridization with target nucleic acid may be polynucleotides or hybridizing analogues or mimetics thereof, including: nucleic acids in which the phosphodiester linkage has been replaced with a substitute linkage, such as phophorothioate, methylimino, methylphosphonate, phosphoramidate, guanidine and the like; nucleic acids in which the ribose subunit has been substituted, e.g., hexose phosphodiester; peptide nucleic acids; and the like. The length of the probes will generally range from 10 to 1000 nts, where in some embodiments the probes will be oligonucleotides and usually range from 15 to 150 nts and more usually from 15 to 100 nts in length, and in other embodiments the probes will be longer, usually ranging in length from 150 to 1000 nts, where the polynucleotide probes may be single- or double-stranded, usually single-stranded, and may be PCR fragments amplified from cDNA.

The probe molecules on the surface of the substrates will correspond to selected genes being analyzed and be positioned on the array at a known location so that positive hybridization events may be correlated to expression of a particular gene in the physiological source from which the target nucleic acid sample is derived. The substrates with which the probe molecules are stably associated may be fabricated from a variety of materials, including plastics, ceramics, metals, gels, membranes, glasses, and the like. The arrays may be produced according to any convenient methodology, such as preforming the probes and then stably associating them with the surface of the support or growing the probes directly on the support. A number of different array configurations and methods for their production are known to those of skill in the art and disclosed in U.S. Pat. Nos. 5,445,934, 5,532,128, 5,556,752, 5,242,974, 5,384,261, 5,405,783, 5,412,087, 5,424,186, 5,429,807, 5,436,327, 5,472,672, 5,527,681, 5,529,756, 5,545,531, 5,554,501, 5,561,071, 5,571,639, 5,593,839, 5,599,695, 5,624,711, 5,658,734, 5,700,637, and 6,004,755.

Following hybridization, where non-hybridized labeled nucleic acid is capable of emitting a signal during the detection step, a washing step is employed where unhybridized labeled nucleic acid is removed from the support surface, generating a pattern of hybridized nucleic acid on the substrate surface. A variety of wash solutions and protocols for their use are known to those of skill in the art and may be used.

Where the label on the target nucleic acid is not directly detectable, one then contacts the array, now comprising bound target, with the other member(s) of the signal producing system that is being employed. For example, where the label on the target is biotin, one then contacts the array with streptavidin-fluorescer conjugate under conditions sufficient for binding between the specific binding member pairs to occur. Following contact, any unbound members of the signal producing system will then be removed, e.g., by washing. The specific wash conditions employed will necessarily depend on the specific nature of the signal producing system that is employed, and will be known to those of skill in the art familiar with the particular signal producing system employed.

The resultant hybridization pattern(s) of labeled nucleic acids may be visualized or detected in a variety of ways, with the particular manner of detection being chosen based on the particular label of the nucleic acid, where representative detection means include scintillation counting, autoradiography, fluorescence measurement, calorimetric measurement, light emission measurement and the like.

Prior to detection or visualization, where one desires to reduce the potential for a mismatch hybridization event to generate a false positive signal on the pattern, the array of hybridized target/probe complexes may be treated with an endonuclease under conditions sufficient such that the endonuclease degrades single stranded, but not double stranded DNA. A variety of different endonucleases are known and may be used, where such nucleases include: mung bean nuclease, S1 nuclease, and the like. Where such treatment is employed in an assay in which the target nucleic acids are not labeled with a directly detectable label, e.g., in an assay with biotinylated target nucleic acids, the endonuclease treatment will generally be performed prior to contact of the array with the other member(s) of the signal producing system, e.g., fluorescent-streptavidin conjugate. Endonuclease treatment, as described above, ensures that only end-labeled target/probe complexes having a substantially complete hybridization at the 3′ end of the probe are detected in the hybridization pattern.

Following hybridization and any washing step(s) and/or subsequent treatments, as described above, the resultant hybridization pattern is detected. In detecting or visualizing the hybridization pattern, the intensity or signal value of the label will be not only be detected but quantified, by which is meant that the signal from each spot of the hybridization will be measured and compared to a unit value corresponding the signal emitted by known number of end-labeled target nucleic acids to obtain a count or absolute value of the copy number of each end-labeled target that is hybridized to a particular spot on the array in the hybridization pattern.

### IV. METHODS OF SCREENING COMPOUNDS FOR NSAID-LIKE ACTIVITY

As discussed above, the present invention provide for screening of compounds for NSAID-like activity. Such assays may comprise random screening of large libraries of candidate substances; alternatively, the assays may be used to focus on particular classes of compounds selected with an eye towards structural attributes that are believed to make them more likely to function like NSAIDs.

To identify an active compound, one generally will examine the expression of various genes in the presence and absence of the candidate substance. For example, a method generally comprises:

(a) providing a cell;

(b) contacting said cell with a candidate compound; and

(c) determining the NSAID's effect on the expression of a plurality of NSAID-modulated genes,

wherein an effect similar to that seen in cells treated with known NSAIDs is predictive that said candidate compound exhibits NSAID-like activity.

It will, of course, be understood that all the screening methods of the present invention are useful in themselves notwithstanding the fact that effective candidates may not be found. The invention provides methods for screening for such candidates, not solely methods of finding them.

As used herein the term “candidate substance” refers to any molecule that may affect the expression of one or more target genes in a manner similar to NSAIDs. The candidate substance may be a protein or fragment thereof, a small molecule, or even a nucleic acid molecule. It may prove to be the case that the most useful pharmacological compounds will be compounds that are structurally related to NSAIDs. Using lead compounds to help develop improved compounds is know as “rational drug design” and includes not only comparisons with know inhibitors and activators, but predictions relating to the structure of target molecules.

The goal of rational drug design is to produce structural analogs of biologically active polypeptides or target compounds. By creating such analogs, it is possible to fashion drugs, which are more active or stable than the natural molecules, which have different susceptibility to alteration or which may affect the function of various other molecules. In one approach, one would generate a three-dimensional structure for a target molecule, or a fragment thereof. This could be accomplished by x-ray crystallography, computer modeling or by a combination of both approaches.

It also is possible to use antibodies to ascertain the structure of a target compound activator or inhibitor. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

On the other hand, one may simply acquire, from various commercial sources, small molecule libraries that are believed to meet the basic criteria for useful drugs in an effort to “brute force” the identification of useful compounds. Screening of such libraries, including combinatorially generated libraries (e.g., peptide libraries), is a rapid and efficient way to screen large number of related (and unrelated) compounds for activity. Combinatorial approaches also lend themselves to rapid evolution of potential drugs by the creation of second, third and fourth generation compounds modeled of active, but otherwise undesirable compounds.

Candidate compounds may include fragments or parts of naturally-occurring compounds, or may be found as active combinations of known compounds, which are otherwise inactive. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds. Thus, it is understood that the candidate substance identified by the present invention may be peptide, polypeptide, polynucleotide, small molecule inhibitors or any other compounds that may be designed through rational drug design starting from known inhibitors or stimulators.

Other suitable modulators include antisense molecules, ribozymes, and antibodies (including single chain antibodies), each of which would be specific for the target molecule. Such compounds are described in greater detail elsewhere in this document. For example, an antisense molecule that bound to a translational or transcriptional start site, or splice junctions, would be ideal candidate inhibitors.

In addition to the modulating compounds initially identified, the inventors also contemplate that other sterically similar compounds may be formulated to mimic the key portions of the structure of the modulators. Such compounds, which may include peptidomimetics of peptide modulators, may be used in the same manner as the initial modulators.

### V. GENE THERAPY

In another embodiment, the present invention provides for the administration of a gene therapy vector encoding one or more genes identified as being upregulated by NSAIDS, or the genetic inhibition of one or more genes that are inhibited by NSAID treatment, for example, by use of antisense, ribozyme or single chain antibody approaches. Various aspects of gene delivery and expression are set forth below.

### 1. Therapeutic Transgenes

Thus, in accordance with the present invention, there are provided methods of treating cancer utilizing genes identified as having increased expression in response to NSAID treatment. Such genes include the genes in the list of “Up-regulated genes by treatment of NSAID s.”

### 2. Inhibiting Transgenes

This technology can be applied against genes that are inhibited by NSAIDs, such as the genes in the list of “Down-regulated genes by treatment of NSAIDs.”

### a. Antisense

The term “antisense” nucleic acid refers to oligo- and polynucleotides complementary to bases sequences of a target DNA or RNA. When introduced into a cell, antisense molecules hybridize to a target nucleic acid and interfere with its transcription, transport, processing, splicing or translation. Targeting double-stranded DNA leads to triple helix formation; targeting RNA will lead to double helix formation.

Antisense constructs may be designed to bind to the promoter or other control regions, exons, introns or even exon-intron boundaries of a gene. Antisense RNA constructs, or DNA encoding such antisense RNA's, may be employed to inhibit gene transcription or translation within a host cell. Nucleic acid sequences which comprise “complementary nucleotides” are those which are capable of base-pairing according to the standard Watson-Crick complementarity rules. That is, that the larger purines will base pair with the smaller pyrimidines to form combinations of guanine paired with cytosine (G:C) and adenine paired with either thymine in the case of DNA (A:T), or uracil (A:U) in the case of RNA. Inclusion of less common bases such as inosine, 5-methylcytosine, 6-methyladenine, hypoxanthine and others in hybridizing sequences does not interfere with pairing.

As used herein, the terms “complementary” and “antisense sequences” mean nucleic acid sequences that are substantially complementary over their entire length and have very few base mismatches. For example, nucleic acid sequences of fifteen bases in length may be termed complementary when they have complementary nucleotides at thirteen or fourteen positions. Naturally, nucleic acid sequences with are “completely complementary” will be nucleic acid sequences which have perfect base pair matching with the target sequences, i.e., no mismatches. Other sequences with lower degrees of homology are contemplated. For example, an antisense construct with limited regions of high homology, but overall containing a lower degree (50% or less) total homology, may be used.

While all or part of the gene sequence may be employed in the context of antisense construction, statistically, any sequence of 17 bases long should occur only once in the human genome and, therefore, suffice to specify a unique target. Although shorter oligomers are easier to make and increase in vivo accessibility, numerous other factors are involved in determining the specificity of hybridization. Both binding affinity and sequence specificity of an oligonucleotide to its complementary target increases with increasing length. It is contemplated that oligonucleotides of 8, 9, 10, 11, 12, 13, 14, 15, 16, 17, 18, 19, 20 or more base pairs will be used. One can readily determine whether a given antisense nucleic acid is effective at targeting a gene simply by testing the construct in vitro to determine whether the gene's function or expression is affected.

In certain embodiments, one may wish to employ antisense constructs which include other elements, for example, those which include C-5 propyne pyrimidines. Oligonucleotides which contain C-5 propyne analogs of uridine and cytidine have been shown to bind RNA with high affinity and to be potent inhibitors or gene expression. Wagner et al. (1993).

### b. Ribozymes

The term “ribozyme” refers to an RNA-based enzyme capable of targeting and cleaving particular DNA and RNA sequences. Ribozymes can either be targeted directly to cells, in the form of RNA oligonucleotides incorporating ribozyme sequences, or introduced into the cell as an expression construct encoding the desired ribozymal RNA. Ribozymes may be used and applied in much the same way as described for antisense nucleic acids. Ribozyme sequences also may be modified in much the same way as described for antisense nucleic acids. For example, one could include modified bases or modified phosphate backbones to improve stability or function.

### C. Single Chain Antibodies

Naturally-occurring antibodies (of isotype IgG) produced by B cells, consist of four polypeptide chains. Two heavy chains (composed of four immunoglobulin domains) and two light chains (made up of two immunoglobulin domains) are held together by disulphide bonds. The bulk of the antibody complex is made up of constant immunoglobulin domains. These have a conserved amino acid sequence, and exhibit low variability. Different classes of constant regions in the stem of the antibody generate different isotypes of antibody with differing properties. The recognition properties of the antibody are carried by the variable regions (VH and VL) at the ends of the arms. Each variable domain contains three hypervariable regions known as complementarity determining regions, or CDRs. The CDRs come together in the final tertiary structure to form an antigen binding pocket. The human genome contains multiple fragments encoding portions of the variable domains in regions of the immunoglobulin gene cluster known as V, D and J. During B cell development these regions undergo recombination to generate a broad diversity of antibody affinities. As these B cell populations mature in the presence of a target antigen, hypermutation of the variable region takes place, with the B cells producing the most active antibodies being selected for further expansion in a process known as affinity maturation.

A major breakthrough was the generation of monoclonal antibodies, pure populations of antibodies with the same affinity. This was achieved by fusing B cells taken from immunized animals with myeloma cells. This generates a population of immortal hybridomas, from which the required clones can be selected. Monoclonal antibodies are very important research tools, and have been used in some therapies. However, they are very expensive and difficult to produce, and if used in a therapeutic context, can elicit and immune response which will destroy the antibody. This can be reduced in part by humanizing the antibody by grafting the CDRs from the parent monoclonal into the backbone of a human IgG antibody. It may be better to deliver antibodies by gene therapy, as this would hopefully provide a constant localized supply of antibody following a single dose of vector. The problems of vector design and delivery are dealt with elsewhere, but antibodies in their native form, consisting of two different polypeptide chains which need to be generated in approximately equal amounts and assembled correctly are not good candidates for gene therapy. However, it is possible to create a single polypeptide which can retain the antigen binding properties of a monoclonal antibody.

The variable regions from the heavy and light chains (VH and VL) are both approximately 110 amino acids long. They can be linked by a 15 amino acid linker (e.g., (glycine₄serine)₃), which has sufficient flexibility to allow the two domains to assemble a functional antigen binding pocket. Addition of various signal sequences allows the scFv to be targeted to different organelles within the cell, or to be secreted. Addition of the light chain constant region (Ck) allows dimerization via disulphide bonds, giving increased stability and avidity. However, there is evidence that scFvs spontaneously multimerize, with the extent of aggregation (presumably via exposed hydrophobic surfaces) being dependent on the length of the glycine-serine linker.

The variable regions for constructing the scFv are obtained as follows. Using a monoclonal antibody against the target of interest, it is a simple procedure to use RT-PCR to clone out the variable regions from mRNA extracted from the parent hybridoma. Degenerate primers targeted to the relatively invariant framework regions can be used. Expression constructs are available with convenient cloning sites for the insertion of the cloned variable regions.

### 2. Vectors

The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques (see, for example, Maniatis et al., 1988 and Ausubel et al., 1994, both incorporated herein by reference).

The term “expression vector” refers to any type of genetic construct comprising a nucleic acid coding for a RNA capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host cell. In addition to control sequences that govern transcription and translation, vectors and expression vectors may contain nucleic acid sequences that serve other functions as well and are described infra.

### a. Promoters and Enhancers

A “promoter” is a control sequence that is a region of a nucleic acid sequence at which initiation and rate of transcription are controlled. It may contain genetic elements at which regulatory proteins and molecules may bind, such as RNA polymerase and other transcription factors, to initiate the specific transcription a nucleic acid sequence. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence.

A promoter generally comprises a sequence that functions to position the start site for RNA synthesis. The best known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Typically, these are located in the region 30-110 bp upstream of the start site, although a number of promoters have been shown to contain functional elements downstream of the start site as well. To bring a coding sequence “under the control of” a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either cooperatively or independently to activate transcription. A promoter may or may not be used in conjunction with an “enhancer,” which refers to a cis-acting regulatory sequence involved in the transcriptional activation of a nucleic acid sequence.

A promoter may be one naturally associated with a nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. For example, promoters that are most commonly used in recombinant DNA construction include the β-lactamase (penicillinase), lactose and tryptophan (trp) promoter systems. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. No. 4,683,202 and 5,928,906, each incorporated herein by reference). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the organelle, cell type, tissue, organ, or organism chosen for expression. Those of skill in the art of molecular biology generally know the use of promoters, enhancers, and cell type combinations for protein expression, (see, for example Sambrook et al. 2000, incorporated herein by reference). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

Additionally any promoter/enhancer combination (as per, for example, the Eukaryotic Promoter Data Base EPDB, http://www.epd.isb-sib.ch/) could also be used to drive expression. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

Table 3 lists non-limiting examples of elements/promoters that may be employed, in the context of the present invention, to regulate the expression of a RNA. Table 4 provides non-limiting examples of inducible elements, which are regions of a nucleic acid sequence that can be activated in response to a specific stimulus.

TABLE 3
Promoter and/or Enhancer

| Promoter/Enhancer | References |
|---|---|
| Immunoglobulin Heavy | Banerji et al., 1983; Gilles et al., 1983; |
| Chain | Grosschedl et al., 1985; Atchinson et al., 1986, |
| | 1987; Imler et al., 1987; Weinberger et al., |
| | 1984; Kiledjian et al., 1988; Porton et al.; 1990 |
| Immunoglobulin Light | Queen et al., 1983; Picard et al., 1984 |
| Chain | |
| T-Cell Receptor | Luria et al., 1987; Winoto et al., 1989; |
| | Redondo et al.; 1990 |
| HLA DQ a and/or DQ β | Sullivan et al., 1987 |
| β-Interferon | Goodbourn et al., 1986; Fujita et al., 1987; |
| | Goodbourn et al., 1988 |
| Interleukin-2 | Greene et al., 1989 |
| Interleukin-2 Receptor | Greene et al., 1989; Lin et al., 1990 |
| MHC Class II 5 | Koch et al., 1989 |
| MHC Class II HLA-Dra | Sherman et al., 1989 |
| β-Actin | Kawamoto et al., 1988; Ng et al.; 1989 |
| Muscle Creatine Kinase | Jaynes et al., 1988; Horlick et al., 1989; |
| (MCK) | Johnson et al., 1989 |
| Prealbumin | Costa et al., 1988 |
| (Transthyretin) | |
| Elastase I | Ornitz et al., 1987 |
| Metallothionein (MTII) | Karin et al., 1987; Culotta et al., 1989 |
| Collagenase | Pinkert et al., 1987; Angel et al., 1987a |
| Albumin | Pinkert et al., 1987; Tronche et al., 1989, 1990 |
| α-Fetoprotein | Godbout et al., 1988; Campere et al., 1989 |
| γ-Globin | Bodine et al., 1987; Perez-Stable et al., 1990 |
| β-Globin | Trudel et al., 1987 |
| c-fos | Cohen et al., 1987 |
| c-HA-ras | Triesman, 1986; Deschamps et al., 1985 |
| Insulin | Edlund et al., 1985 |
| Neural Cell Adhesion | Hirsch et al., 1990 |
| Molecule (NCAM) | |
| α₁-Antitrypsin | Latimer et al., 1990 |
| H2B (TH2B) Histone | Hwang et al., 1990 |
| Mouse and/or Type I | Ripe et al., 1989 |
| Collagen | |
| Glucose-Regulated Pro- | Chang et al., 1989 |
| teins (GRP94 and | |
| GRP78) | |
| Rat Growth Hormone | Larsen et al., 1986 |
| Human Serum Amyloid | Edbrooke et al., 1989 |
| A (SAA) | |
| Troponin I (TN I) | Yutzey et al., 1989 |
| Platelet-Derived Growth | Pech et al., 1989 |
| Factor (PDGF) | |
| Duchenne Muscular | Klamut et al., 1990 |
| Dystrophy | |
| SV40 | Banerji et al., 1981; Moreau et al., 1981; Sleigh |
| | et al., 1985; Firak et al., 1986; Herr et al., |
| | 1986; Imbra et al., 1986; Kadesch et al., 1986; |
| | Wang et al., 1986; Ondek et al., 1987; Kuhl |
| | et al., 1987; Schaffner et al., 1988 |
| Polyoma | Swartzendruber et al., 1975; Vasseur et al., |
| | 1980; Katinka et al., 1980, 1981; Tyndell et al., |
| | 1981; Dandolo et al., 1983; de Villiers et al., |
| | 1984; Hen et al., 1986; Satake et al., 1988; |
| | Campbell and/or Villarreal, 1988 |
| Retroviruses | Kriegler et al., 1982, 1983; Levinson et al., |
| | 1982; Kriegler et al., 1983, 1984a, b, 1988; |
| | Bosze et al., 1986; Miksicek et al., 1986; |
| | Celander et al., 1987; Thiesen et al., 1988; |
| | Celander et al., 1988; Choi et al., 1988; |
| | Reisman et al., 1989 |
| Papilloma Virus | Campo et al., 1983; Lusky et al., 1983; |
| | Spandidos and/or Wilkie, 1983; Spalholz et al., |
| | 1985; Lusky et al., 1986; Cripe et al., 1987; |
| | Gloss et al., 1987; Hirochika et al., 1987; |
| | Stephens et al., 1987 |
| Hepatitis B Virus | Bulla et al., 1986; Jameel et al., 1986; Shaul |
| | et al., 1987; Spandau et al., 1988; Vannice |
| | et al., 1988 |
| Human Inimuno- | Muesing et al., 1987; Hauber et al., 1988; |
| deficiency Virus | Jakobovits et al., 1988; Feng et al., 1988; |
| | Takebe et al., 1988; Rosen et al., 1988; |
| | Berkhout et al., 1989; Laspia et al., 1989; |
| | Sharp et al., 1989; Braddock et al., 1989 |
| Cytomegalovirus (CMV) | Weber et al., 1984; Boshart et al., 1985; |
| | Foecking et al., 1986 |
| Gibbon Ape Leukemia | Holbrook et al., 1987; Quinn et al., 1989 |
| Virus | |

TABLE 4
Inducible Elements

| Element | Inducer | References |
|---|---|---|
| MT II | Phorbol Ester (TFA) | Palmiter et al., 1982; |
| | Heavy metals | Haslinger et al., 1985; |
| | | Searle et al., 1985; Stuart |
| | | et al., 1985; Imagawa |
| | | et al., 1987, Karin et al., |
| | | 1987; Angel et al., |
| | | 1987b; MeNeall et al., |
| | | 1989 |
| MMTV (mouse mam- | Glucocorticoids | Huang et al., 1981; Lee |
| mary tumor virus) | | et al., 1981; Majors et |
| | | al., 1983; Chandler et al., |
| | | 1983; Lee et al., 1984; |
| | | Ponta et al., 1985; Sakai |
| | | et al., 1988 |
| β-Interferon | Poly(rI)x | Tavernier et al., 1983 |
| | Poly(rc) | |
| Adenovirus 5 E2 | ElA | Imperiale et al., 1984 |
| Collagenase | Phorbol Ester (TPA) | Angel et al., 1987a |
| Stromelysin | Phorbol Ester (TPA) | Angel et al., 1987b |
| SV40 | Phorbol Ester (TPA) | Angel et al., 1987b |
| Murine MX Gene | Interferon, Newcastle | Hug et al., 1988 |
| | Disease Virus | |
| GRP78 Gene | A23187 | Resendez et al., 1988 |
| α-2-Macroglobulin | IL-6 | Kunz et al., 1989 |
| Vimentin | Serum | Rittling et al., 1989 |
| MHC Class I Gene | Interferon | Blanar et al., 1989 |
| H-2Kb | | |
| HSP70 | ElA, SV40 Large T | Taylor et al., 1989, |
| | Antigen | 1990a, 1990b |
| Proliferin | Phorbol Ester-TPA | Mordacq et al., 1989 |
| Tumor Necrosis | PMA | Hensel et al., 1989 |
| Factor α | | |
| Thyroid Stimulating | Thyroid Hormone | Chatterjee et al., 1989 |
| Hormone α Gene | | |

The identity of tissue-specific promoters or elements, as well as assays to characterize their activity, is well known to those of skill in the art. Non-limiting examples of such regions include the human LIMK2 gene (Nomoto et al., 1999), the somatostatin receptor 2 gene (Kraus et al., 1998), murine epididymal retinoic acid-binding gene (Lareyre et al., 1999), human CD4 (Zhao-Emonet et al., 1998), mouse alpha2 (XI) collagen (Tsumaki et al., 1998), D1A dopamine receptor gene (Lee et al., 1997), insulin-like growth factor II (Wu et al., 1997), and human platelet endothelial cell adhesion molecule-1 (Almendro et al., 1996).

### b. Initiation Signals and Internal Ribosome Binding Sites

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments of the invention, the use of internal ribosome entry sites (IRES) elements are used to create multigene, or polycistronic, messages. IRES elements are able to bypass the ribosome scanning model of 5′-methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, each herein incorporated by reference).

### c. Multiple Cloning Sites

Vectors can include a multiple cloning site (MCS), which is a nucleic acid region that contains multiple restriction enzyme sites, any of which can be used in conjunction with standard recombinant technology to digest the vector (see, for example, Carbonelli et al., 1999, Levenson et al., 1998, and Cocea, 1997, incorporated herein by reference.) “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

### d. Splicing Sites

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression (see, for example, Chandler et al., 1997, herein incorporated by reference).

### e. Termination Signals

The vectors or constructs of the present invention will generally comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels.

In eukaryotic systems, the terminator region may also comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to more stable and are translated more efficiently. Thus, in other embodiments involving eukaryotes, it is preferred that that terminator comprises a signal for the cleavage of the RNA, and it is more preferred that the terminator signal promotes polyadenylation of the message. The terminator and/or polyadenylation site elements can serve to enhance message levels and to minimize read through from the cassette into other sequences.

Terminators contemplated for use in the invention include any known terminator of transcription described herein or known to one of ordinary skill in the art, including but not limited to, for example, the termination sequences of genes, such as for example the bovine growth hormone terminator or viral termination sequences, such as for example the SV40 terminator. In certain embodiments, the termination signal may be a lack of transcribable or translatable sequence, such as due to a sequence truncation.

### f. Polyadenylation Signals

In expression, particularly eukaryotic expression, one will typically include a polyadenylation signal to effect proper polyadenylation of the transcript. The nature of the polyadenylation signal is not believed to be crucial to the successful practice of the invention, and any such sequence may be employed. Preferred embodiments include the SV40 polyadenylation signal or the bovine growth hormone polyadenylation signal, convenient and known to function well in various target cells. Polyadenylation may increase the stability of the transcript or may facilitate cytoplasmic transport.

### g. Origins of Replication

In order to propagate a vector in a host cell, it may contain one or more origins of replication sites (often termed “ori”), which is a specific nucleic acid sequence at which replication is initiated. Alternatively an autonomously replicating sequence (ARS) can be employed if the host cell is yeast.

### h. Selectable and Screenable Markers

In certain embodiments of the invention, cells containing a nucleic acid construct of the present invention may be identified in vitro or in vivo by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as GFP, whose basis is calorimetric analysis, are also contemplated. Alternatively, screenable enzymes such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selectable and screenable markers are well known to one of skill in the art.

### i. Plasmid Vectors

In certain embodiments, a plasmid vector is contemplated for use to transform a host cell. In general, plasmid vectors containing replicon and control sequences which are derived from species compatible with the host cell are used in connection with these hosts. The vector ordinarily carries a replication site, as well as marking sequences which are capable of providing phenotypic selection in transformed cells. In a non-limiting example, E. coli is often transformed using derivatives of pBR322, a plasmid derived from an E. coli species. pBR322 contains genes for ampicillin and tetracycline resistance and thus provides easy means for identifying transformed cells. The pBR plasmid, or other microbial plasmid or phage must also contain, or be modified to contain, for example, promoters which can be used by the microbial organism for expression of its own proteins.

In addition, phage vectors containing replicon and control sequences that are compatible with the host microorganism can be used as transforming vectors in connection with these hosts. For example, the phage lambda GEM™-11 may be utilized in making a recombinant phage vector which can be used to transform host cells, such as, for example, E. coli LE392.

Further useful plasmid vectors include pIN vectors (Inouye et al., 1985); and pGEX vectors, for use in generating glutathione S-transferase (GST) soluble fusion proteins for later purification and separation or cleavage. Other suitable fusion proteins are those with β-galactosidase, ubiquitin, and the like.

Bacterial host cells, for example, E. coli, comprising the expression vector, are grown in any of a number of suitable media, for example, LB. The expression of the recombinant protein in certain vectors may be induced, as would be understood by those of skill in the art, by contacting a host cell with an agent specific for certain promoters, e.g., by adding IPTG to the media or by switching incubation to a higher temperature. After culturing the bacteria for a further period, generally of between 2 and 24 h, the cells are collected by centrifugation and washed to remove residual media.

### j. Viral Vectors

The ability of certain viruses to infect cells or enter cells via receptor-mediated endocytosis, and to integrate into host cell genome and express viral genes stably and efficiently have made them attractive candidates for the transfer of foreign nucleic acids into cells (e.g., mammalian cells). Non-limiting examples of virus vectors that may be used to deliver a nucleic acid of the present invention are described below.

### 1. Adenoviral Vectors

A particular method for delivery of the nucleic acid involves the use of an adenovirus expression vector. Although adenovirus vectors are known to have a low capacity for integration into genomic DNA, this feature is counterbalanced by the high efficiency of gene transfer afforded by these vectors. “Adenovirus expression vector” is meant to include those constructs containing adenovirus sequences sufficient to (a) support packaging of the construct and (b) to ultimately express a tissue or cell-specific construct that has been cloned therein. Knowledge of the genetic organization or adenovirus, a 36 kb, linear, double-stranded DNA virus, allows substitution of large pieces of adenoviral DNA with foreign sequences up to 7 kb (Grunhaus and Horwitz, 1992).

### 2. AAV Vectors

The nucleic acid may be introduced into the cell using adenovirus assisted transfection. Increased transfection efficiencies have been reported in cell systems using adenovirus coupled systems (Kelleher and Vos, 1994; Cotten et al., 1992; Curiel, 1994). Adeno-associated virus (AAV) is an attractive vector system as it has a high frequency of integration and it can infect non-dividing cells, thus making it useful for delivery of genes into mammalian cells, for example, in tissue culture (Muzyczka, 1992) or in vivo. AAV has a broad host range for infectivity (Tratschin et al., 1984; Laughlin et al., 1986; Lebkowski et al., 1988; McLaughlin et al., 1988). Details concerning the generation and use of rAAV vectors are described in U.S. Pat. Nos. 5,139,941 and 4,797,368, each incorporated herein by reference.

### 3. Retroviral Vectors

Retroviruses have promise as gene delivery vectors due to their ability to integrate their genes into the host genome, transferring a large amount of foreign genetic material, infecting a broad spectrum of species and cell types and of being packaged in special cell-lines (Miller, 1992).

In order to construct a retroviral vector, a nucleic acid (e.g., one encoding gene of interest) is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into a special cell line (e.g., by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

Lentiviruses are complex retroviruses, which, in addition to the common retroviral genes gag, pol, and env, contain other genes with regulatory or structural function. Lentiviral vectors are well known in the art (see, for example, Naldini et al., 1996; Zufferey et al., 1997; Blomer et al., 1997; U.S. Pat. Nos. 6,013,516 and 5,994,136). Some examples of lentivirus include the Human Immunodeficiency Viruses: HIV-1, HIV-2 and the Simian Immunodeficiency Virus: SIV. Lentiviral vectors have been generated by multiply attenuating the HIV virulence genes, for example, the genes env, vif, vpr, vpu and nef are deleted making the vector biologically safe.

Recombinant lentiviral vectors are capable of infecting non-dividing cells and can be used for both in vivo and ex vivo gene transfer and expression of nucleic acid sequences. For example, recombinant lentivirus capable of infecting a non-dividing cell wherein a suitable host cell is transfected with two or more vectors carrying the packaging functions, namely gag, pol and env, as well as rev and tat is described in U.S. Pat. No. 5,994,136, incorporated herein by reference. One may target the recombinant virus by linkage of the envelope protein with an antibody or a particular ligand for targeting to a receptor of a particular cell-type. By inserting a sequence (including a regulatory region) of interest into the viral vector, along with another gene which encodes the ligand for a receptor on a specific target cell, for example, the vector is now target-specific.

### 4. Other Viral Vectors

Other viral vectors may be employed as vaccine constructs in the present invention. Vectors derived from viruses such as vaccinia virus (Ridgeway, 1988; Baichwal and Sugden, 1986; Coupar et al., 1988), sindbis virus, cytomegalovirus and herpes simplex virus may be employed. They offer several attractive features for various mammalian cells (Friedmann, 1989; Ridgeway, 1988; Baichwal and Sugden, 1986; Coupar et al., 1988; Horwich et al., 1990).

### k. Delivery Using Modified Viruses

A nucleic acid to be delivered may be housed within an infective virus that has been engineered to express a specific binding ligand. The virus particle will thus bind specifically to the cognate receptors of the target cell and deliver the contents to the cell. A novel approach designed to allow specific targeting of retrovirus vectors was developed based on the chemical modification of a retrovirus by the chemical addition of lactose residues to the viral envelope. This modification can permit the specific infection of hepatocytes via sialoglycoprotein receptors.

Another approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

### l. Vector Delivery and Cell Transformation

Suitable methods for nucleic acid delivery for transformation of an organelle, a cell, a tissue or an organism for use with the current invention are believed to include virtually any method by which a nucleic acid (e.g., DNA) can be introduced into an organelle, a cell, a tissue or an organism, as described herein or as would be known to one of ordinary skill in the art. Such methods include, but are not limited to, direct delivery of DNA such as by ex vivo transfection (Wilson et al., 1989, Nabel et al, 1989), by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harlan and Weintraub, 1985; U.S. Pat. No. 5,789,215, incorporated herein by reference); by electroporation (U.S. Pat. No. 5,384,253, incorporated herein by reference; Tur-Kaspa et al., 1986; Potter et al., 1984); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991) and receptor-mediated transfection (Wu and Wu, 1987; Wu and Wu, 1988); by microprojectile bombardment (PCT Application Nos. WO 94/09699 and 95/06128; U.S. Pat. Nos. 5,610,042; 5,322,783 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium-mediated transformation (U.S. Pat. Nos. 5,591,616 and 5,563,055, each incorporated herein by reference); by PEG-mediated transformation of protoplasts (Omirulleh et al., 1993; U.S. Pat. Nos. 4,684,611 and 4,952,500, each incorporated herein by reference); by desiccation/inhibition-mediated DNA uptake (Potrykus et al., 1985), and any combination of such methods. Through the application of techniques such as these, organelle(s), cell(s), tissue(s) or organism(s) may be stably or transiently transformed.

### 1. Injection

In certain embodiments, a nucleic acid may be delivered to an organelle, a cell, a tissue or an organism via one or more injections (i.e., a needle injection), such as, for example, subcutaneously, intradermally, intramuscularly, intravenously, intraperitoneally, etc. Methods of injection of vaccines are well known to those of ordinary skill in the art (e.g., injection of a composition comprising a saline solution). Further embodiments of the present invention include the introduction of a nucleic acid by direct microinjection. Direct microinjection has been used to introduce nucleic acid constructs into Xenopus oocytes (Harland and Weintraub, 1985).

### 2. Electroporation

In certain embodiments of the present invention, a nucleic acid is introduced into an organelle, a cell, a tissue or an organism via electroporation. Electroporation involves the exposure of a suspension of cells and DNA to a high-voltage electric discharge. In some variants of this method, certain cell wall-degrading enzymes, such as pectin-degrading enzymes, are employed to render the target recipient cells more susceptible to transformation by electroporation than untreated cells (U.S. Pat. No. 5,384,253, incorporated herein by reference). Alternatively, recipient cells can be made more susceptible to transformation by mechanical wounding.

Transfection of eukaryotic cells using electroporation has been quite successful. Mouse pre-B lymphocytes have been transfected with human kappa-immunoglobulin genes (Potter et al., 1984), and rat hepatocytes have been transfected with the chloramphenicol acetyltransferase gene (Tur-Kaspa et al., 1986) in this manner.

### 3. Calcium Phosphate

In other embodiments of the present invention, a nucleic acid is introduced to the cells using calcium phosphate precipitation. Human KB cells have been transfected with adenovirus 5 DNA (Graham and Van Der Eb, 1973) using this technique. Also in this manner, mouse L(A9), mouse C127, CHO, CV-1, BHK, NIH3T3 and HeLa cells were transfected with a neomycin marker gene (Chen and Okayama, 1987), and rat hepatocytes were transfected with a variety of marker genes (Rippe et al., 1990).

### 4. DEAE-Dextran

In another embodiment, a nucleic acid is delivered into a cell using DEAE-dextran followed by polyethylene glycol. In this manner, reporter plasmids were introduced into mouse myeloma and erythroleukemia cells (Gopal, 1985).

### 5. Sonication Loading

Additional embodiments of the present invention include the introduction of a nucleic acid by direct sonic loading. LTK- fibroblasts have been transfected with the thymidine kinase gene by sonication loading (Fechheimer et al., 1987).

### 6. Liposome-Mediated Transfection

In a further embodiment of the invention, a nucleic acid may be entrapped in a lipid complex such as, for example, a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated is an nucleic acid complexed with Lipofectamine (Gibco BRL) or Superfect (Qiagen).

Liposome-mediated nucleic acid delivery and expression of foreign DNA in vitro has been very successful (Nicolau and Sene, 1982; Fraley et al., 1979; Nicolau et al., 1987). The feasibility of liposome-mediated delivery and expression of foreign DNA in cultured chick embryo, HeLa and hepatoma cells has also been demonstrated (Wong et al., 1980).

In certain embodiments of the invention, a liposome may be complexed with a hemagglutinating virus (HVJ). This has been shown to facilitate fusion with the cell membrane and promote cell entry of liposome-encapsulated DNA (Kaneda et al., 1989). In other embodiments, a liposome may be complexed or employed in conjunction with nuclear non-histone chromosomal proteins (HMG-1) (Kato et al, 1991). In yet further embodiments, a liposome may be complexed or employed in conjunction with both HVJ and HMG-1. In other embodiments, a delivery vehicle may comprise a ligand and a liposome.

### 7. Receptor Mediated Transfection

Still further, a nucleic acid may be delivered to a target cell via receptor-mediated delivery vehicles. These take advantage of the selective uptake of macromolecules by receptor-mediated endocytosis that will be occurring in a target cell. In view of the cell type-specific distribution of various receptors, this delivery method adds another degree of specificity to the present invention.

Certain receptor-mediated gene targeting vehicles comprise a cell receptor-specific ligand and a nucleic acid-binding agent. Others comprise a cell receptor-specific ligand to which the nucleic acid to be delivered has been operatively attached. Several ligands have been used for receptor-mediated gene transfer (Wu and Wu, 1987; Wagner et al., 1990; Perales et al., 1994; Myers, EPO 0273085), which establishes the operability of the technique. Specific delivery in the context of another mammalian cell type has been described (Wu and Wu, 1993; incorporated herein by reference). In certain aspects of the present invention, a ligand will be chosen to correspond to a receptor specifically expressed on the target cell population.

In other embodiments, a nucleic acid delivery vehicle component of a cell-specific nucleic acid targeting vehicle may comprise a specific binding ligand in combination with a liposome. The nucleic acid(s) to be delivered are housed within the liposome and the specific binding ligand is functionally incorporated into the liposome membrane. The liposome will thus specifically bind to the receptor(s) of a target cell and deliver the contents to a cell. Such systems have been shown to be functional using systems in which, for example, epidermal growth factor (EGF) is used in the receptor-mediated delivery of a nucleic acid to cells that exhibit upregulation of the EGF receptor.

In still further embodiments, the nucleic acid delivery vehicle component of a targeted delivery vehicle may be a liposome itself, which will preferably comprise one or more lipids or glycoproteins that direct cell-specific binding. For example, lactosyl-ceramide, a galactose-terminal asialganglioside, have been incorporated into liposomes and observed an increase in the uptake of the insulin gene by hepatocytes (Nicolau et al., 1987). It is contemplated that the tissue-specific transforming constructs of the present invention can be specifically delivered into a target cell in a similar manner.

### 8. Microprojectile Bombardment

Microprojectile bombardment techniques can be used to introduce a nucleic acid into at least one, organelle, cell, tissue or organism (U.S. Pat. Nos. 5,550,318, 5,538,880, 5,610,042, and PCT Application WO 94/09699; each of which is incorporated herein by reference). This method depends on the ability to accelerate DNA-coated microprojectiles to a high velocity allowing them to pierce cell membranes and enter cells without killing them (Klein et al., 1987). There are a wide variety of microprojectile bombardment techniques known in the art, many of which are applicable to the invention.

Microprojectile bombardment may be used to transform various cell(s), tissue(s) or organism(s), such as for example any plant species. Examples of species which have been transformed by microprojectile bombardment include monocot species such as maize (PCT Application WO 95/06128), barley (Ritala et al., 1994; Hensgens et al., 1993), wheat (U.S. Pat. No. 5,563,055, incorporated herein by reference), rice (Hensgens et al., 1993), oat (Torbet et al., 1995; Torbet et al., 1998), rye (Hensgens et al., 1993), sugarcane (Bower et al., 1992), and sorghum (Casas et al., 1993; Hagio et al., 1991); as well as a number of dicots including tobacco (Tomes et al., 1990; Buising and Benbow, 1994), soybean (U.S. Pat. No. 5,322,783, incorporated herein by reference), sunflower (Knittel et al. 1994), peanut (Singsit et al., 1997), cotton (McCabe and Martinell, 1993), tomato (VanEck et al. 1995), and legumes in general (U.S. Pat. No. 5,563,055, incorporated herein by reference).

In this microprojectile bombardment, one or more particles may be coated with at least one nucleic acid and delivered into cells by a propelling force. Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold particles or beads. Exemplary particles include those comprised of tungsten, platinum, and preferably, gold. It is contemplated that in some instances DNA precipitation onto metal particles would not be necessary for DNA delivery to a recipient cell using microprojectile bombardment. However, it is contemplated that particles may contain DNA rather than be coated with DNA. DNA-coated particles may increase the level of DNA delivery via particle bombardment but are not, in and of themselves, necessary.

For the bombardment, cells in suspension are concentrated on filters or solid culture medium. Alternatively, immature embryos or other target cells may be arranged on solid culture medium. The cells to be bombarded are positioned at an appropriate distance below the macroprojectile stopping plate.

An illustrative embodiment of a method for delivering DNA into a cell (e.g., a plant cell) by acceleration is the Biolistics Particle Delivery System, which can be used to propel particles coated with DNA or cells through a screen, such as a stainless steel or Nytex screen, onto a filter surface covered with cells, such as for example, a monocot plant cells cultured in suspension. The screen disperses the particles so that they are not delivered to the recipient cells in large aggregates. It is believed that a screen intervening between the projectile apparatus and the cells to be bombarded reduces the size of projectiles aggregate and may contribute to a higher frequency of transformation by reducing the damage inflicted on the recipient cells by projectiles that are too large.

### VI. PHARMACEUTICAL FORMULATIONS AND ROUTES OF ADMINISTRATION

Where clinical applications are contemplated, it will be necessary to prepare pharmaceutical compositions in a form appropriate for the intended application. Generally, this will entail preparing compositions that are essentially free of pyrogens, as well as other impurities that could be harmful to humans or animals.

The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Supplementary active ingredients also can be incorporated into the compositions.

Administration of these compositions according to the present invention will be via any common route so long as the target tissue is available via that route. This includes intradermal, subcutaneous, intramuscular, intraperitoneal or intravenous injection. In particular, intratumoral routes and sites local and regional to tumors are contemplated. Such compositions would normally be administered as pharmaceutically acceptable compositions, described supra.

The active compounds also may be administered parenterally or intraperitoneally. Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy administration by a syringe is possible. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

For oral administration the polypeptides of the present invention may be incorporated with excipients that may include water, binders, abrasives, flavoring agents, foaming agents, and humectants.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The compositions of the present invention may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

### VII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

Cell culture. The HCA-7 cells, which are derived from a rectal carcinoma, were kindly provided by Dr. Susan Kirkland (ICRF, London, UK). Cells were cultured at 37° C. with 5% CO₂ in McCoy's 5A medium containing penicillin, streptomycin, 10% FBS (Life Technologies, Inc., Grand Island, N.Y.). NS-398 and nimesulide (Cayman chemicals, Ann Arbor, Mich.), sulindac sulfide (Merck & Co., Rahway, N.J.) and SC-58125 (G.D. Searle Co., St. Louis, Mo.) were dissolved in DMSO prior to addition to cell culture medium. Experimental controls were treated with 0.1% DMSO.

mRNA isolation. Total cellular RNA was isolated using Tri-Reagent (Molecular Research Center, Inc., Cincinnati, Ohio) and polyadenylated RNA was isolated using a mRNA Purification Kit (Amersham Pharmacia Biotech, Inc., Piscataway, N.J.).

Suppression subtractive hybridization. SSH was performed with the PCR-Select™ cDNA Subtraction Kit (Clontech Laboratories Inc., Palo Alto, Calif.) as described by the manufacturer except for slight modifications of the PCR and hybridization conditions. Starting material consisted of 2 μg of mRNA from NS-398 treated HCA-7 cells and 2 μg of mRNA from control cells. After synthesis of the first and second cDNA strands, the cDNA was digested with Rsa I to obtain shorter, blunt-ended molecules. Then ligation of two different adapters to the cDNA fragments was performed separately. The adapter sequences were as follows: 5′-CTAATACGACTCACTATAGGGCTCGAGCGGCCGCCCGGGCAGGT-3′ (adapter 1-SEQ ID NO: 52) and 5′-CTAATACGACTCACTATAGGGCAGCGTGGTCGCGGCCGAGGT-3′ (adapter 2R-SEQ ID NO: 53). For the first round hybridization, the mixture of driver and tester cDNA were denatured at 98° C. for 1.5 min and then maintained at 68° C. for ˜10 h. For the second round hybridization, driver cDNA was denatured at 98° C. for 1.5 min and then added to the mixture of the two previous hybridization and allowed to incubate at 68° C. for ˜18 h.

After two rounds of hybridization, two rounds of PCR were performed to amplify the specific, differentially expressed cDNA fragments. All PCR reactions were performed using a PTC-100 thermal cycler (MJ Research, Inc. Watertown, Mass.). The primer used in first round is 5′-CTAATACGACTCACTATAGGGC-3′ (primer 1-SEQ ID NO: 54) and the primers used in second round were 5′-TCGAGCGGCCGCCCGGGCAGGT-3′ (nested primer 1-SEQ ID NO: 55) and 5′-AGCGTGGTCGCGGCCGAGGT-3′ (nested primer 2R -SEQ ID NO: 56). Cycling parameters are as follows: for the first round, 27 cycles of 94° C. for 30 s, 64° C. for 30 s and 71° C. for 1.5 min; for the second round, 15 cycles of 94° C. for 30 s, 68° C. for 30 s and 72° C. for 1.5 min.

To evaluate subtractive efficiency, the same amount of subtracted and unsubtracted secondary PCR products were applied as templates, and GAPDH primers were used to perform PCR. The PCR products were examined in a 2.0% agarose/EtBr gel to detect the abundance of GAPDH. After evaluation of the subtractive efficiency, the subtracted PCR products were cloned directly into the pCR2.1-TOPO vector (InVitrogen, Carlsbad, Calif.). Plates were incubated at 37° C. until small clones were visible and the blue/white staining could be clearly distinguished.

Differential screening. Differential screening was performed by using the PCR-Select™ Differential Screening Kit (Clontech) according to the users manual. After picking the recombinant clones from the subtractive library and identifying them by restriction digestion, the insert was PCR amplified in 20 μl volume reactions using nested PCR primer 1 and 2R. After amplification, 5 μl of the PCR product was denatured with 5 μl of 0.6 N NaOH and then 1.5 μl of each mixture was transferred to a nylon membrane. Each cDNA fragment was arrayed in duplicate in each blot and two identical blots were prepared for hybridization with different probes. The total secondary PCR amplified products from the forward and reverse SSH were used as probes. After washing, the membranes were exposed overnight to Kodak BioMax MR x-ray film (Eastman Kodak Company, Rochester, N.Y.) with and intensifying screen at −70° C.

Northern blotting. Total cellular RNA samples (˜30 μg/lane) were denatured and fractionated in 1% formaldehyde-agarose gels and then blotted to nylon filters. The blots were hybridized with cDNA probes labeled with α-[³²P] dCTP by using a Prime-It random prime labeling kit (Stratagene, La Jolla, Calif.). A GAPDH probe was used as an internal control to determine the variability of loading among lanes.

Sequencing analysis. Sequencing of cDNA inserts was accomplished in the DNA Sequencing Core Lab of Vanderbilt-Ingram Cancer Center. Sequence homology searches were conducted using BLAST program against the NCBI databases (www.ncbi.nim.nih.gov).

### Example 2

### Results

The inventors found significant inhibition of HCA-7 cell growth following treatment with NS-398. To evaluate this process more carefully, they employed SSH to study changes in gene expression following treatment. Cells were treated with 100 μM of NS-398 for 4 days and then harvested for mRNA isolation. SSH and DS were performed. To isolate sequences which are downregulated following NS-398 treatment, tester cDNA, prepared from drug treated mRNA, and excess driver cDNA from control mRNA samples (forward subtraction) were used. To recover sequences upregulated upon NS-398 treatment, control cDNA as tester and cDNA from drug treated sample as driver (reverse subtraction) were used. Using SSH, a total of 57 cDNAs were identified. Twenty-eight clones were obtained from the forward subtraction and twenty-nine clones from the reverse subtraction.

After hybridization, the subtraction efficiency was measured using PCR to amplify GAPDH, a known housekeeping gene. Two rounds of subtraction had a very high efficiency of removing background sequences. The GAPDH band appeared after 33 cycles with the subtracted products as template, while a signal was detected after only 18 cycles using template from the non-subtracted samples.

In general, the SSH technique had some background that could lead to false positives. Therefore, randomly picking clones from the subtracted library for Northern blot analysis would be time consuming and inefficient. It was found that performing the DS step helps minimize the background before embarking on an evaluation of the expression status of each individual gene. To perform DS, all the duplicate clones were arrayed in each filter and the filter was hybridized separately with the total secondary PCR amplified products from the forward and reverse SSH as probes. A clone was classified as positive only when it showed a greater than 3- to 4-fold difference in expression.

Forty-two differentially expressed cDNAs were identified and their sequence compared to those reported in the available GenBank/EMBL databases online. Twenty-three novel cDNA fragments were found, while 19 clones had some homology to gene sequences reported in the database. These 19 clones were evaluated by Northern blot analysis of total RNA obtained from treated and control HCA-7 cells. Of these, 12 clones were differentially expressed (Table 5).

TABLE 5
Gene fragments differentially expressed following NS-398 treatment^{a}

| Accession | Size | | Regulation |
|---|---|---|---|
| Number | (bp) | Identity | by NS-398^{b,c,d} |
| AF098158 | 263 | Proliferation associated p100 | down (2.2) |
| | | protein | |
| D14657 | 720 | KIAA0101 | down (2.7) |
| AF060515 | 482 | Cyclin K | down (2.1) |
| AB06757 | 749 | PCDH7 (BH-Pcdh) | up (26.0) |
| NM_011050 | 515 | Pdcd4 | up (1.5) |
| | | (Programmed cell death) | |
| NM_005245 | 466 | FAT tumor suppressor | up (4.4) |
| | | (Drosophila) homolog | |
| NM_006457 | 498 | ENH, LIM protein (similar to rat | up (3.0) |
| | | protein kinase C-binding enigma) | |
| U22816 | 809 | LIP.1 (LAR-interacting protein 1b) | up (3.4) |
| NM_004945 | 386 | Dynamin 2 | up (3.1) |

^{a}the differential expression of all the genes listed was confirmed by Differential Screening and Northern Blotting
^{b}“down” indicates decreased levels following treatment
^{c}“up” indicates increased levels following treatment
^{d}the number in parentheses is the -fold increase or decrease after treatment

Among the genes that were down-regulated by NS-398, one was designated as NRG-1 (NSAID Regulated Gene 1), which is also known as KIAA0101 (Nagase et al., 1995). Additional COX-2 inhibitors have been developed and are reported to have anti-neoplastic effects. To determine if NRG-1 is regulated by other NSAIDs, the effects of SC-58125, sulindac sulfide, and nemisulide on gene expression in HCA-7 cells were measured. All of these agents reduced NRG-1 mRNA levels from 3 to 20 fold. The inventors also evaluated the expression of NRG-1 in human colon cancers and matched normal tissues. Among the six pairs of human colon cancers and the related normal mucosal samples, five pairs showed high levels of NRG-1 expression in colon carcinomas compared to adjacent normal mucosa.

### Example 3

### Discussion

Among the known genes identified, some belong to the cadherin superfamily, which is a family of single-transmembrane calcium-dependent cell adhesion proteins. Some of the genes identified regulate cell cycle progression and several are involved in signaling pathways which control apoptosis and differentiation.

Human FAT (Mahoney et al., 1991) and PCDH7 (Yoshida et al., 1998), which are up-regulated following treatment, are members of the cadherin family. Defining mechanisms involved in cadherin-mediated cell-cell adhesion is at the core of understanding a diverse range of cellular phenomena. The human FAT gene is homologous to Drosophila fat, a tumor suppressor gene which is essential for controlling cell proliferation during Drosophila development. Mutations in the FAT gene lead to a cell autonomous overgrowth phenotype (Dunne et al., 1995).

Cyclin K was found to be down-regulated following treatment with NS-398. This is a new member of the “transcription” cyclin family and most closely related to human cyclin C and H. It was reported that cyclin K plays a dual role in regulating Cdk and RNAP-2 activity (Edwards et al., 1998). Another gene involved in the regulation of cell proliferation is the p100 gene, which codes for a novel proliferation-associated nuclear protein. P100 protein is exclusively expressed in proliferating cells and specifically restricted to the S, G2, and M phase off the cell cycle (Heidebrecht et al., 1997).

Pdcd4, Dynamin 2 and LIP.1 are all up-regulated by NS-398 in HCA-7 cells. Pdcd4 is homologous to a mouse apoptosis related gene, MA-3. MA-3 was found in many apoptosis-inducible cell lines, including thymocytes, T cells, B cells and pheochromocytoma (Shibahara et al., 1995). More recently, Cmarik et al. reported that the pdcd4 protein inhibits neoplastic transformation in mouse JB6 cells (Cmarik et al., 1999). Dynamin 2 is a ubiquitously expressed isoform of dynamin which is a member of the GTPase superfamily. Fish et al. reported that an increase of dynamin 2 activates p53 and induces apoptosis in a p53-dependent fashion (Fish et al., 2000). LIP.1 is a cytoplasmic 160 kD phosphoserine protein which binds to the LAR membrane-distal D2 protein tyrosine phosphatase domain and appears to localize at focal adhesions. LAR is a widely expressed receptor-like protein tyrosine phosphatase that is implicated in regulation of intracellular signaling triggered by both cell adhesion and peptide growth factors (Serra-Pages et al., 1995). Wang et al. indicated that LAR can inhibit FGF-induced MAPK activation by inhibiting the tyrosine phosphorylation of signal transducers that act downstream of the FGF receptor and the overexpression of LAR in mammalian cells induces caspase-dependent apoptosis (Wang et al., 2000; Weng et al., 1999).

ENH is a LIM protein whose levels are increased following NS-398 treatment. LIM contains a cysteine-rich domain composed of two independent zinc-finger domains which has been proposed to direct protein-protein interactions. ENH contains three LIM domains in the C-terminal region and was shown to be associated with PKC in an isoform specific manner in vivo and was phosphorylated by PKC in vitro (Kuroda et al., 1996). The function of ENH is not clear, but recently Nakagawa et al. reported that ENH may play an important role in heart development (Nakagawa et al., 2000).

NRG-1, reported as KIAA0101, was first cloned from a cDNA library made from human KG-1 myeloid cells (Nagase et al., 1995). NRG-1 contains an open reading frame encoding 111 amino acids that has no homology to any known protein. In this study, the inventors demonstrated that NRG-1 is down regulated following treatment with several different NSAIDs and its levels are higher in human colon cancers compared to normal tissue. NRG-1 is also induced by K-ras in rat intestinal epithelial cells (data not shown). NRG-1 may represent a downstream gene regulated by NSAIDs that is involved in anti-neoplastic effects of NSAIDs.

An anti NRG-1 antibody (polyclonal antibody A26) prepared from rabbit antiserum was generated using a synthetic peptide H-CGTYRKVVAARAPRKVL-OH (SEQ ID NO: 57). The synthetic peptide corresponded to amino acids 11-26 which map near the amino terminus of human NRG-1 protein. The synthetic peptide was conjugated to a carrier such as keyhole limpet haemocyanin (KLH). The purity of the peptide was found to be 100% by HPLC analysis. Female NZW rabbits were injected with the immunogen several times and the serum collected and tested by ELISA.

Whole tissue lysates from normal tissue and human colon cancer tissue were analyzed for NRG protein expression by Western Blotting. In brief, 20 μg of whole tissue lysates was separated by SDS-PAGE (4-20%) and transferred to nitrocellulose membrane. After incubating the membrane overnight in blocking solution, it was probed with anti-NRG serum (1:500 dilution) for 3 hours. The membrane was then washed three times and the second antibody (anti-rabbit) was added for 1 hour. The membrane was washed three times and developed by the ECL chemiluminescence system. Among the 15 pairs of tissue samples compared, there were 12 pairs which showed an increased expression of NRG protein in tumor tissue than the normal mucosa..

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

U.S. Pat. No. 4,683,195

U.S. Pat. No. 4,683,202

U.S. Pat. No. 4,684,611

U.S. Pat. No. 4,797,368

U.S. Pat. No. 4,800,159

U.S. Pat. No. 4,883,750

U.S. Pat. No. 4,952,500

U.S. Pat. No. 5,139,941

U.S. Pat. No. 5,242,974

U.S. Pat. No. 5,279,721

U.S. Pat. No. 5,302,523

U.S. Pat. No. 5,322,783

U.S. Pat. No. 5,384,253

U.S. Pat. No. 5,384,261

U.S. Pat. No. 5,405,783

U.S. Pat. No. 5,412,087

U.S. Pat. No. 5,424,186

U.S. Pat. No. 5,429,807

U.S. Pat. No. 5,436,327

U.S. Pat. No. 5,445,934

U.S. Pat. No. 5,464,765

U.S. Pat. No. 5,472,672

U.S. Pat. No. 5,527,681

U.S. Pat. No. 5,529,756

U.S. Pat. No. 5,532,128

U.S. Pat. No. 5,538,877

U.S. Pat. No. 5,538,880

U.S. Pat. No. 5,545,531

U.S. Pat. No. 5,550,318

U.S. Pat. No. 5,554,501

U.S. Pat. No. 5,556,752

U.S. Pat. No. 5,561,071

U.S. Pat. No. 5,563,055

U.S. Pat. No. 5,571,639

U.S. Pat. No. 5,580,859

U.S. Pat. No. 5,589,466

U.S. Pat. No. 5,591,616

U.S. Pat. No. 5,593,839

U.S. Pat. No. 5,599,695

U.S. Pat. No. 5,610,042

U.S. Pat. No. 5,610,042

U.S. Pat. No. 5,624,711

U.S. Pat. No. 5,656,610

U.S. Pat. No. 5,658,734

U.S. Pat. No. 5,700,637

U.S. Pat. No. 5,702,932

U.S. Pat. No. 5,736,524

U.S. Pat. No. 5,780,448

U.S. Pat. No. 5,789,215

U.S. Pat. No. 5,814,625

U.S. Pat. No. 5,840,873

U.S. Pat. No. 5,843,640

U.S. Pat. No. 5,843,650

U.S. Pat. No. 5,843,651

U.S. Pat. No. 5,843,663

U.S. Pat. No. 5,843,929

U.S. Pat. No. 5,846,708

U.S. Pat. No. 5,846,709

U.S. Pat. No. 5,846,717

U.S. Pat. No. 5,846,726

U.S. Pat. No. 5,846,729

U.S. Pat. No. 5,846,783

U.S. Pat. No. 5,849,481

U.S. Pat. No. 5,849,486

U.S. Pat. No. 5,849,487

U.S. Pat. No. 5,849,497

U.S. Pat. No. 5,849,546

U.S. Pat. No. 5,849,547

U.S. Pat. No. 5,851,772

U.S. Pat. No. 5,853,990

U.S. Pat. No. 5,853,992

U.S. Pat. No. 5,853,993

U.S. Pat. No. 5,856,092

U.S. Pat. No. 5,858,652

U.S. Pat. No. 5,861,244

U.S. Pat. No. 5,863,732

U.S. Pat. No. 5,863,753

U.S. Pat. No. 5,866,331

U.S. Pat. No. 5,866,366

U.S. Pat. No. 5,882,864

U.S. Pat. No. 5,900,481

U.S. Pat. No. 5,905,024

U.S. Pat. No. 5,910,407

U.S. Pat. No. 5,912,124

U.S. Pat. No. 5,912,145

U.S. Pat. No. 5,912,148

U.S. Pat. No. 5,916,776

U.S. Pat. No. 5,916,779

U.S. Pat. No. 5,919,626

U.S. Pat. No. 5,919,630

U.S. Pat. No. 5,922,574

U.S. Pat. No. 5,925,517

U.S. Pat. No. 5,925,565

U.S. Pat. No. 5,928,862

U.S. Pat. No. 5,928,869

U.S. Pat. No. 5,928,905

U.S. Pat. No. 5,928,906

U.S. Pat. No. 5,929,227

U.S. Pat. No. 5,932,413

U.S. Pat. No. 5,932,451

U.S. Pat. No. 5,935,791

U.S. Pat. No. 5,935,819

U.S. Pat. No. 5,935,825

U.S. Pat. No. 5,939,291

U.S. Pat. No. 5,942,391

U.S. Pat. No. 5,945,100

U.S. Pat. No. 5,981,274

U.S. Pat. No. 5,994,136

U.S. Pat. No. 5,994,136

U.S. Pat. No. 5,994,624

U.S. Pat. No. 6,004,755

U.S. Pat. No. 6,013,516

Akopian and Wood, J. Biol. Chem., 270:21264-21270, 1995.

Almendro et al., J. Iminunol., 157:5411-5421, 1996.

Angel et al., Mol. Cell. Biol., 7:2256, 1987a.

Angel et al., Cell, 49:729, 1987b.

Atchison and Perry, Cell, 46:253, 1986.

Atchison and Perry, Cell, 48:121, 1987.

Ausubel et al., In: Current Protocols in Molecular Biology, John, Wiley & Sons, Inc, New York, 1994.

Baichwal and Sugden, In: Gene Transfer, Kucherlapati (ed.), New York, Plenum Press, 117-148, 1986.

Banerji et al., Cell, 27(2 Pt 1):299-308, 1981.

Baneiji et al., Cell, 33(3):729-740, 1983.

Berkhout et al., Cell, 59:273-282, 1989.

Blanar et al., EMBO J., 8:1139, 1989.

Blomer et al., J. Virol., 71(9):6641-6649, 1997.

Bodine and Ley, EMBO J., 6:2997, 1987.

Boshart et al., Cell, 41:521, 1985.

Bosze et al., EMBO J., 5(7):1615-1623, 1986.

Bower et al., The Plant Journal, 2:409-416. 1992.

Braddock et al., Cell, 58:269, 1989.

Buising and Benbow, Mol. Gen. Genet., 243(1):71-81. 1994.

Bulla and Siddiqui, J. Virol., 62:1437, 1986.

Campbell and Villarreal, Mol. Cell. Biol., 8:1993, 1988.

Campere and Tilghman, Genes and Dev., 3:537, 1989.

Campo et al., Nature, 303:77, 1983.

Carbonelli et al., FEMS Microbiol, Lett., 177(1):75-82, 1999.

Casas et al., Proc. Natl. Acad. Sci. USA, 90(23):11212-11216, 1993.

Celander and Haseltine, J. Virology, 61:269, 1987.

Celander et al., J. Virology, 62:1314, 1988.

Chandler et al., Cell, 33:489, 1983.

Chandler et al., Proc. Natl. Acad. Sci. USA, 94(8):3596-601, 1997.

Chang et al., Mol. Cell. Biol., 20:8571-8579, 2000.

Chang et al., Mol. Cell. Biol., 9:2153, 1989.

Chatterjee et al., Proc. Natl. Acad. Sci. USA, 86:9114, 1989.

Chen and Okayama, Mol. Cell. Biol., 7(8):2745-2752, 1987.

Choi et al., Cell, 53:519, 1988.

Cmarik et al., Proc. Natl. Acad. Sci. USA, 96:14037-14042, 1999.

Cocea, Biotechniques, 23(5):814-816, 1997.

Cohen et al, J. Cell. Physiol., 5:75, 1987.

Costa et al., Mol. Cell. Biol., 8:81, 1988.

Cotton et al., Proc. Natl. Acad. Sci. USA, 89(13):6094-6098, 1992.

Coupar et al., Gene, 68:1-10, 1988.

Cripe et al., EMBO J., 6:3745, 1987.

Culotta and Hamer, Mol. Cell. Biol., 9:1376, 1989.

Curiel, Nat. Immun,, 13(2-3):141-64, 1994.

Dandolo et al., J. Virology, 47:55-64, 1983.

De Villiers et al., Nature, 312(5991):242-246, 1984.

Deleersnijder et al., J. Biol. Chem., 271:19475-9482, 1996.

Deschamps et al., Science, 230:1174-1177, 1985.

Diatchenko et al., Proc. Natl. Acad. Sci. USA, 93:6025-6030, 1996.

Drmanac and Drmanac, Methods Enzymol., 303:165-178, 1999.

DuBois, Radhika, Reddy, Entingh, Gastroenterology, 110:1259-1262, 1996.

Dunne et al., Genomics, 30:207-223, 1995.

Eberhart et al., Gastroenterology, 107:1183-1188, 1994.

Edbrooke et al., Mol. Cell. Biol., 9:1908, 1989.

Edlund et al., Science, 230:912-916, 1985.

Edwards et al., Mol. Cell. Biol., 18:4291-4300, 1998.

European Application No. 0273085

European Application No. 320308

European Application No. 329822

Fechheimer et al., Proc. Natl Acad. Sci. USA, 84:8463-8467, 1987.

Feng and Holland, Nature, 334:6178, 1988.

Firak and Subramanian, Mol. Cell. Biol., 6:3667, 1986.

Fish et al., J. Cell. Biol., 150:145-154, 2000.

Foecking and Hofstetter, Gene, 45(1):101-105, 1986.

Fraley et al., Proc. Natl Acad. Sci. USA, 76:3348-3352, 1979.

Friedmann, Science, 244:1275-1281, 1989.

Frohman, In: PCR Protocols: A Guide To Methods And Applications, Academic Press, N.Y., 1990.

Fujita et al., Cell, 49:357, 1987.

GB Application No. 2 202 328

Ghosh and Bachhawat, In: Liver Diseases, Targeted Diagnosis and Therapy Using Specific Receptors and Ligands. Wu et al., eds., Marcel Dekker, New York, 87-104, 1991.

Gilles et al., Cell, 33:717, 1983.

Gloss et al., EMBO J., 6:3735, 1987.

Godbout et al., Mol. Cell. Biol., 8:1169, 1988.

Goodbourn and Maniatis, Proc. Natl Acad. Sci. USA, 85:1447, 1988.

Goodbourn et al., Cell, 45:601, 1986.

Gopal, Mol. Cell. Biol., 5:1188-1190, 1985.

Greene et al., Immunology Today, 10:272, 1989

Grosschedl and Baltimore, Cell, 41:885, 1985.

Grunhaus et al., Seminar in Virology, 200(2):535-546, 1992.

Hagio et al., Plant Cell Rep., 10(5):260-264, 1991.

Hanif et al., Biochem. Pharmacol., 52:237-245, 1996.

Hara et al., Jpn. J. Cancer Res., 88:600-604, 1997.

Harland and Weintraub, J. Cell Biol., 101:1094-1099, 1985.

Haslinger and Karin, Proc. Natl Acad. Sci. USA, 82:8572, 1985.

Hauber and Cullen, J. Virology, 62:673, 1988.

He et al., Cell, 99:335-345, 1999.

Heidebrecht et al., Blood, 90:226-233, 1997.

Hen et al., Nature, 321:249, 1986.

Hensel et al., Lymphokine Res., 8:347, 1989.

Hensgens et al., Plant Mol. Biol., 22(6):1101-1127, 1993.

Herr and Clarke, Cell, 45:461, 1986.

Hirochika et al., J. Virol., 61:2599, 1987.

Hirsch et al., Mol. Cell. Biol., 10:1959, 1990.

Holbrook et al., Virology, 157:211, 1987.

Horlick and Benfield, Mol. Cell. Biol., 9:2396, 1989.

Horwich et al., Virol., 64:642-650, 1990.

Huang et al., Cell, 27:245, 1981.

Hug et al., Mol. Cell. Biol., 8:3065, 1988.

Hwang et al., Mol. Cell. Biol., 10:585, 1990.

Imagawa et al., Cell, 51:251, 1987.

Imbra and Karin, Nature, 323:555, 1986.

Imler et al., Mol. Cell Biol., 7:2558, 1987.

Imperiale and Nevins, Mol. Cell. Biol., 4:875, 1984.

Innis et al., Proc. Natl. Acad. Sci. USA, 85(24):9436-9440, 1988.

Inouye and Inouye, Nucleic Acids Res., 13: 3101-3109, 1985.

Jacoby et al., Cancer Res., 60:5040-5044, 2000.

Jakobovits et al., Mol. Cell. Biol., 8:2555, 1988.

Jameel and Siddiqui, Mol. Cell Biol., 6:710, 1986.

Jaynes et al., Mol. Cell. Biol., 8:62, 1988.

Johnson et al., Mol. Cell. Biol., 9:3393, 1989.

Jones et al., Nat. Med., 5:1418-1423, 1999.

Kadesch and Berg, Mol. Cell. Biol., 6:2593, 1986.

Kaeppler et al., Plant Cell Reports 9:415-418, 1990.

Kaneda et al., Science, 243:375-378, 1989.

Karin et al., Mol. Cell. Biol., 7:606, 1987.

Karin et al., Mol. Cell. Biol., 7:606, 1987.

Katinka et al., Cell, 20:393, 1980.

Kato et al, J. Biol. Chem., 266:3361-3364, 1991.

Kawamoto et al., Mol. Cell. Biol., 8:267, 1988.

Kelleher and Vos, Biotechniques, 17(6):1110-7, 1994.

Kiledjian et al., Mol. Cell. Biol., 8:145, 1988.

Kinoshita et al., Biochim. Biophys. Acta., 1438:120-130, 1999.

Klamut et al., Mol. Cell. Biol., 10:193, 1990.

Klein et al., Nature, 327:70-73, 1987.

Knittel et al., Plant Cell Reports, 14(2-3):81-86, 1994.

Koch et al., Mol. Cell. Biol., 9:303, 1989.

Kopp and Ghosh, Science, 265:956-959, 1994.

Kraus et al., FEBS Lett., 428(3):165-170, 1998.

Kriegler and Botchan, In: Eukaryotic Viral Vectors, Gluzman (Ed), Cold Spring Harbor Laboratory, NY, 1982.

Kriegler and Botchan, Mol. Cell. Biol., 3:325, 1983.

Kriegler et al., Cell, 38:483, 1984.

Kriegler et al., Cell, 53:45, 1988.

Kuang et al., Nucleic Acids Res., 26:1116-1123, 1998.

Kuhl et al., Cell, 50:1057, 1987.

Kunz et al., Nucl. Acids Res., 17:1121, 1989.

Kuroda et al., J. Biol. Chem., 271:31029-31032, 1996.

Kwoh et al., Proc. Nat. Acad. Sci. USA, 86:1173, 1989.

Landis et al., CA. Cancer J. Clin., 49:8-31, 1999.

Lareyre et al., J. Biol. Chem., 274(12):8282-8290, 1999.

Larsen et al., Proc. Natl. Acad. Sci. USA, 83:8283, 1986.

Laspia et al., Cell, 59:283, 1989.

Latimer et al., Mol. Cell. Biol., 10:760, 1990.

Laughlin et al., J. Virol,, 60(2):515-524, 1986.

Lebkowski et al., Mol. Cell. Biol., 8(10):3988-3996, 1988.

Lee et al., DNA Cell. Biol,, 16(11):1267-1275, 1997.

Lee et al., Nature, 294:228, 1981.

Lee et al., Nucleic Acids Res., 12:4191-206, 1984.

Lehmann et al., J. Biol. Chem., 272:3406-3410, 1997.

Levenson et al., Hum. Gene Ther., 9(8):1233-6, 1998.

Levinson et al., Nature, 295:79, 1982.

Liang and Pardee, Science, 257:967-971, 1992.

Lin et al., Mol. Cell. Biol., 10:850, 1990.

Lisitsyn, Lisitsyn, Wigler, Science, 259:946-951, 1993.

Liu et al., Cancer Res., 58:4245-4249, 1998.

Liu et al., J. Urol., 164:820-825, 2000.

Luria et al., EMBO J., 6:3307, 1987.

Lusky and Botchan, Proc. Natl. Acad. Sci. USA, 83:3609, 1986.

Lusky et al., Mol. Cell. Biol. 3:1108, 1983.

Macejak and Sarnow, Nature, 353:90-94, 1991.

Mahoney et al., Cell, 67:853-868, 1991.

Majors and Varmus, Proc. Natl. Acad. Sci. USA, 80:5866, 1983.

Mann et al., Cell, 33:153-159, 1983.

Maser and Calvet, Semin. Nephrol., 15:29-42, 1995.

McCabe and Martinell, Bio-Technology, 11(5):596-598, 1993.

McGinty et al., J. Biol. Chem., 275:12095-12101, 2000.

McLaughlin et al., J. Virol., 62(6):1963-1973, 1988.

McNeall et al., Gene, 76:81, 1989.

Miksicek et al., Cell, 46:203, 1986.

Miller et al., Am. J. Clin. Oncol., 15(3):216-221, 1992.

Mordacq and Linzer, Genes and Dev., 3:760, 1989.

Moreau et al., Nucl. Acids Res., 9:6047, 1981.

Muesing et al., Cell, 48:691, 1987.

Muzyczka, Curr. Top Microbiol. Immunol., 158:97-129, 1992.

Nabel et al, Science, 244(4910):1342-1344, 1989.

Nagase et al., DNA Res., 2:51-59, 1995.

Nakagawa et al., Biochem. Biophys. Res. Commun., 272:505-512, 2000.

Naldini et al., Science, 272(5259):263-267, 1996.

Ng et al., Nuc. Acids Res., 17:601, 1989.

Nicolas and Rubenstein, In: Vectors: A survey of molecular cloning vectors and their uses, Rodriguez and Denhardt (eds.), Stoneham: Butterworth, pp. 493-513, 1988.

Nicolau and Sene, Biochim. Biophys. Acta., 721:185-190, 1982.

Nicolau et al., Methods Enzymol., 149:157-176, 1987.

Nishimura et al., Jpn. J. Cancer Res., 90:1152-1162, 1999.

Nomoto et al., Gene, 236(2):259-271, 1999.

Ohara et al., Proc. Natl Acad. Sci. USA, 86:5673-5677, 1989.

Omirulleh et al., Plant Mol. Biol., 21(3):415-28, 1993.

Ondek et al., EMBO J., 6:1017, 1987.

Omitz et al., Mol. Cell. Biol., 7:3466, 1987.

Oshima et al., Cell, 87:803-809, 1996.

Palmiter et al., Nature, 300:611, 1982.

Paskind et al., Virology, 67:242-248, 1975.

PCT Application No. WO 87/00880

PCT Application No. WO 88/10315

PCT Application No. WO 89/01025

PCT Application No. WO 89/06700

PCT Application No. WO 90/07641

PCT Application No. WO 94/09699

PCT Application No. WO 94/09699

PCT Application No. WO 95/06128

Pech et al., Mol. Cell. Biol., 9:396, 1989.

Pelletier and Sonenberg, Nature, 334:320-325, 1988.

Perales et al., Proc. Natl Acad. Sci. USA, 91:4086-4090, 1994.

Perez-Stable and Constantini, Mol. Cell. Biol., 10: 1116, 1990.

Physician's Desk Reference, Medical Economics Data, Montville, N.J., 1745-1747, 1999

Picard and Schaffier, Nature, 307:83, 1984.

Pinkert et al., Genes and Dev., 1:268, 1987.

Ponta et al., Proc. Natl. Acad. Sci. USA, 82:1020, 1985.

Porton et al., Mol. Cell. Biol., 10:1076, 1990.

Potrykus et al., Mol. Gen. Genet., 199(2):169-77, 1985.

Potter et al., Proc. Natl Acad. Sci. USA, 81:7161-7165, 1984.

Potter et al., Proc. Natl Acad. Sci. USA, 81:7161-7165, 1984.

Queen and Baltimore, Cell, 35:741, 1983.

Quinn et al., Mol. Cell. Biol., 9:4713, 1989.

Redondo et al., Science, 247:1225, 1990.

Reisman and Rotter, Mol. Cell. Biol., 9:3571, 1989.

Resendez Jr. et al., Mol. Cell. Biol., 8:4579, 1988.

Ridgeway, In: Vectors: A survey of molecular cloning vectors and their uses, Stoneham: Butterworth, 467-492, 1988.

Rioux and Castonguay, Cancer Res., 58:5354-5360, 1998.

Ripe et al., Mol. Cell. Biol., 9:2224, 1989.

Rippe et al., Mol. Cell. Biol., 10:689-695, 1990.

Ritala et al., Plant Mol. Biol., 24(2):317-325, 1994.

Rittling et al., Nuc. Acids Res., 17:1619, 1989.

Rosen et al., Cell, 41:813, 1988.

Roux et al., Proc. Natl Acad. Sci. USA, 86:9079-9083, 1989.

Sakai et al., Genes and Dev., 2:1144, 1988.

Sambrook et al., In: Molecular cloning, Cold Spring Harbor Laboratory Press, Cold Spring Harbor, NY, 2000.

Satake et al., J. Virology, 62:970, 1988.

Sawaoka et al., Am. J. Physiol., 274:G1061-G1067, 1998.

Schaffner et al., J. Mol. Biol., 201:81, 1988.

Searle et al., Mol. Cell. Biol., 5:1480, 1985.

Serra-Pages et al., Embo. J., 14:2827-2838, 1995.

Sharp and Marciniak, Cell, 59:229, 1989.

Shaul and Ben-Levy, EMBO J., 6:1913, 1987.

Sheng et al., J. Clin. Invest., 99:2254-2259, 1997.

Sherman et al., Mol. Cell. Biol., 9:50, 1989.

Shibahara et al., Gene, 166:297-301, 1995.

Shureiqi et al., J. Natl. Cancer Inst., 92:1136-1142, 2000.

Singsit et al., Transgenic Res., 6(2):169-176, 1997.

Sleigh and Lockett, J. EMBO, 4:3831, 1985.

Smith et al., Eur. J. Cancer, 36:664-674, 2000.

Spalholz et al., Cell, 42:183, 1985.

Spandau and Lee, J. Virology, 62:427, 1988.

Spandidos and Wilkie, EMBO J., 2:1193, 1983.

Stephens and Hentschel, Biochem. J., 248:1, 1987.

Stuart et al., Nature, 317:828, 1985.

Sullivan and Peterlin, Mol. Cell. Biol., 7:3315, 1987.

Swartzendruber and Lehman, J. Cell. Physiology, 85:179, 1975.

Takebe et al., Mol. Cell. Biol., 8:466, 1988.

Tavernier et al., Nature, 301:634, 1983.

Taylor and Kingston, Mol. Cell. Biol., 10:165, 1990a.

Taylor and Kingston, Mol. Cell. Biol., 10:176, 1990b.

Taylor et al., J. Biol. Chem., 264:15160, 1989.

Temin, In: Gene Transfer, Kucherlapati (ed.), New York: Plenum Press, pp. 149-188, 1986.

Thiesen et al, J. Virology, 62:614, 1988.

Thun et al., N. Engl. J. Med., 325:1593-1596, 1991.

Tomes et al., Plant. Mol. Biol., 14(2):261-268, 1990.

Torbet et al., Crop Science, 38(1):226-231, 1998.

Torbet et al., Plant Cell Reports, 14(10):635-640, 1995.

Tratschin et al., Mol. Cell. Biol., 4:2072-2081, 1984.

Treisman, Cell, 42:889, 1985.

Tronche et al., Mol. Biol. Med., 7:173, 1990.

Trudel and Constantini, Genes and Dev. 6:954, 1987.

Tsuji et al., Prostaglandins Leukot. Essent. Fatty Acids, 55:179-183, 1996.

Tsujii and DuBois, Cell, 83:493-501, 1995.

Tsujii et al., Cell, 93:705-716, 1998.

Tsujii et al., Proc. Natl. Acad. Sci. USA, 94:3336-3340, 1997.

Tsumaki et al., J. Biol. Chem., 273(36):22861-22864, 1998.

Tur-Kaspa et al., Mol. Cell. Biol., 6:716-718, 1986.

Tyndell et al., Nuc. Acids. Res., 9:6231, 1981.

Van Eck et al., Plant Cell Reports, 14(5):299-304, 1995.

Vannice and Levinson, J. Virology, 62:1305, 1988.

Vasseur et al., Proc. Natl. Acad. Sci. USA, 77:1068, 1980.

von Stein et al., Nucleic Acids Res., 25:2598-2602, 1997.

Wagner et al., Proc. Natl. Acad. Sci. USA 87(9):3410-3414, 1990.

Wagner et al., Science, 260:1510-1513, 1993.

Walker et al., Proc. Natl. Acad. Sci. USA, 89:392-396 1992.

Wang and Calame, Cell, 47:241, 1986.

Wang et al., Oncogene, 19:2346-2353, 2000.

Watanabe et al., Cancer Res., 59:5093-5096, 1999.

Weber et al., Cell, 36:983, 1984.

Wechter et al., Cancer Res., 57:4316-4324, 1997.

Weinberger et al., Mol. Cell. Biol., 8:988, 1984.

Weng et al., Genes Cells, 4:185-196, 1999.

Williams et al., Cancer Res., 60:6045-6051, 2000b.

Williams et al., Gastroenterology, 111:1134-1140, 1996.

Williams et al., J. Clin. Invest., 105:1589-1594, 2000a.

Wilson et al., Science, 244:1344-1346, 1989.

Winoto and Baltimore, Cell, 59:649, 1989.

Wong et al., Gene, 10:87-94, 1980.

Wu and Wu, Adv. Drug Delivery Rev., 12:159-167, 1993.

Wu and Wu, Biochemistry, 27:887-892, 1988.

Wu and Wu, J. Biol. Chem., 262:4429-4432, 1987.

Wu et al., Biochem. Biophys. Res. Commun., 233(1):221-226, 1997.

Yang et al., Proc. Natl. Acad. Sci. USA, 87:9568-9572, 1990.

Yin et al., Nature, 396:77-80, 1998.

Yoshida et al., Genomics, 49:458-461, 1998.

Yoshimi et al., Jpn. J. Cancer Res., 90:406-412, 1999.

Yutzey et al. Mol. Cell.. Biol., 9:1397, 1989.

Zeng et al., Nucleic Acids Res., 22:4381-4385, 1994.

Zhang et al., J. Exp. Med., 190:451-459, 1999.

Zhao-Emonet et al., Biochem. Biophys. Acta., 1442(2-3):109-19, 1998.

Zuber et al., Nat. Genet., 24:144-152, 2000.

Zufferey et al., Nat. Biotechnol., 15(9):871-875, 1997.

What is claimed is:

1. A method for predicting the response of a cancer cell to treatment with an NSAID comprising:
(a) providing a cancer cell;
(b) contacting said cancer cell with an NSAID; and
(c) determining the NSAID's effect on the expression of a plurality of NSAID-modulated genes,
wherein an effect similar to that seen in NSAID-responsive cancer cells is predictive that said cancer cell also would be responsive to NSAID treatment.

2. The method of claim 1, wherein said cancer cell is selected from the group consisting of a brain cancer cell, a head & neck cancer cell, an esophageal cancer cell, a lung cancer cell, a thyroid cancer cell, a stomach cancer cell, a colon cancer cell, a liver cancer cell, a kidney cancer cell, a prostate cancer cell, a breast cancer cell, a cervical cancer cell, an ovarian cancer cell, a testicular cancer cell, a rectal cancer cell, a skin cancer cell or a blood cancer cell.

3. The method of claim 1, wherein said NSAID is a non-steroidal anti-inflammatory drug.

4. The method of claim 1, wherein said plurality of NSAID-modulated genes comprises at least one gene selected from the group consisting of the genes listed in Table 1 and Table 2.

5. The method of claim 4, wherein said plurality of NSAID-modulated genes comprises at least one gene selected from the group consisting of SEQ. ID. NO: 1, SEQ. ID. NO: 2, SEQ. ID. NO: 3, SEQ. ID. NO: 6, SEQ. ID. NO: 7, SEQ. ID. NO: 18, SEQ. ID. NO: 31, SEQ. ID. NO: 32, SEQ. ID. NO: 33, SEQ. ID. NO: 38, SEQ. ID. NO: 39, or SEQ. ID. NO: 42.

6. The method of claim 4, wherein the plurality comprises at least two genes selected from the group consisting of the genes listed in Table 1 and Table 2.

7. The method of claim 7, wherein the plurality comprises at least three genes selected from the group consisting of the genes listed in Table 1 and Table 2.

8. The method of claim 7, wherein the plurality comprises at least five genes selected from the group consisting of the genes listed in Table 1 and Table 2.

9. The method of claim 4, wherein the plurality comprises at least one gene that is not listed in Table 1 or Table 2.

10. The method of claim 1, wherein the plurality comprises at least one gene listed in Table 5.

11. The method of claim 1, wherein determining comprises quantitating a mRNA for NSAID-modulated genes.

12. The method of claim 11, wherein quantitating comprises Northern analysis.

13. The method of claim 11, wherein quantitating comprises densitometric scanning of a one- or two-dimensional gel.

14. The method of claim 11, wherein quantitating comprises nucleic acid amplification.

15. The method of claim 14, wherein nucleic acid amplification comprises quantitative RT-PCR.

16. The method of claim 11, wherein quantitating comprises nucleic acid hybrizidation.

17. The method of claim 14, wherein hybridization comprises a nucleic acid array.

18. The method of claim 17, wherein said nucleic acid array is disposed on a chip or wafer.

19. The method of claim 18, wherein said chip is a glass or membrane that carries nucleic acid.

20. The method of claim 1, wherein determining comprises quantitating NSAID-modulated polypeptides.

21. The method of claim 20, wherein quantitating comprises densitometric scanning of a one- or two-dimensional gel.

22. The method of claim 20, wherein quantitating comprises immunologic detection of said polypeptides.

23. The method of claim 22, wherein immunologic detection comprises Western blot, ELISA, or RIA.

24. The method of claim 1, wherein said cancer cell is obtained from a subject in need of cancer therapy.

25. The method of claim 24, further comprising treating said subject.

26. The method of claim 1, further comprising treating a cancer cell known to be responsive to said NSAID with said NSAID, and comparing the expression of the same plurality of NSAID-modulated genes with the results of step (c).

27. A method for predicting the efficacy of an NSAID in cancer therapy comprising:
(a) providing a cancer cell;
(b) contacting said cancer cell with an NSAID; and
(c) determining the NSAID's effect on the expression of a plurality of NSAID-modulated genes,
wherein an effect similar to that seen in NSAID-responsive cancer cells treated with known cancer-therapeutic NSAIDs is predictive that said NSAID also would be efficacious in cancer therapy.

28. The method of claim 27, wherein said cancer cell is selected from the group consisting of a brain cancer cell, a head & neck cancer cell, an esophageal cancer cell, a lung cancer cell, a thyroid cancer cell, a stomach cancer cell, a colon cancer cell, a liver cancer cell, a kidney cancer cell, a prostate cancer cell, a breast cancer cell, a cervical cancer cell, an ovarian cancer cell, a testicular cancer cell, a rectal cancer cell, a skin cancer cell or a blood cancer cell.

29. The method of claim 27, wherein said NSAID is a non-steroidal anti-inflammatory drug.

30. The method of claim 27, wherein said plurality of NSAID-modulated genes are selected from the group consisting of the genes listed in Table 1 and Table 2.

31. The method of claim 30, wherein the plurality comprises at least one gene selected from the group consisting of the genes listed in Table 1 and Table 2.

32. The method of claim 31, wherein said plurality of genes comprises at least one gene selected from the group consisting of SEQ. ID. NO: 1, SEQ. ID. NO: 2, SEQ. ID. NO: 3, SEQ. ID. NO: 6, SEQ. ID. NO: 7, SEQ. ID. NO: 18, SEQ. ID. NO: 31, SEQ. ID. NO: 32, SEQ. ID. NO: 33, SEQ. ID. NO: 38, SEQ. ID. NO: 39, or SEQ. ID. NO: 42.

33. The method of claim 30, wherein the plurality comprises at least two genes selected from the group consisting of the genes listed in Table 1 and Table 2.

34. The method of claim 33, wherein the plurality comprises at least three genes selected from the group consisting of the genes listed in Table 1 and Table 2.

35. The method of claim 34, wherein the plurality comprises at least five genes selected from the group consisting of the genes listed in Table 1 and Table 2.

36. The method of claim 30, wherein the plurality comprises at least one gene that is not listed in Table 1 or Table 2.

37. The method of claim 27, wherein the plurality comprises at least one gene listed in Table 5.

38. The method of claim 27, wherein determining comprises quantitating a mRNA for NSAID-modulated genes.

39. The method of claim 38, wherein quantitating comprises Northern analysis.

40. The method of claim 38, wherein quantitating comprises densitometric scanning of a one- or two-dimensional gel.

41. The method of claim 38, wherein quantitating comprises nucleic acid amplification.

42. The method of claim 41, wherein nucleic acid amplification comprises quantitative RT-PCR.

43. The method of claim 38, wherein quantitating comprises nucleic acid hybrizidation.

44. The method of claim 41, wherein hybridization comprises a nucleic acid array.

45. The method of claim 44, wherein said nucleic acid array is disposed on a chip or wafer.

46. The method of claim 45, wherein said chip is a glass or membrane that carries nucleic acid.

47. The method of claim 27, wherein determining comprises quantitating NSAID-modulated polypeptides.

48. The method of claim 47, wherein quantitating comprises densitometric scanning of a one- or two-dimensional gel.

49. The method of claim 47, wherein quantitating comprises immunologic detection of said polypeptides.

50. The method of claim 49, wherein immunologic detection comprises Western blot, ELISA, or RIA.

51. The method of claim 27, wherein said cancer cell is obtained from a subject in need of cancer therapy.

52. The method of claim 51, further comprising treating said subject.

53. The method of claim 27, further comprising treating a cancer cell, known to be responsive to said NSAID, with said NSAID, and comparing the expression of the same plurality of NSAID-modulated genes with the results of step (c).

54. The method of claim 53, wherein the NSAID is aspirin, sulindac or celecoxib.

55. A method for treating a subject with cancer comprising administering to said subject a composition that modulates the levels of one or more of NSAID-modulated polypeptides.

56. The method of claim 55, wherein said composition increases the level of one or more of NSAID-stimulated polypeptides.

57. The method of claim 56, wherein said one or more NSAID-stimulated polypeptides are translation products of the NSAID-stimulated genes.

58. The method of claim 55, wherein said composition decreases the level of one or more of NSAID-inhibited polypeptides.

59. The method of claim 58, wherein said one or more NSAID-inhibited polypeptides are translation products of the NSAID-inhibited genes.

60. The method of claim 55, further comprising administering to said subject two compositions, wherein a first composition increases the level of one or more of NSAID-stimulated polypeptides, and a second composition decreases the level of one or more of NSAID-inhibited polypeptides.

61. The method of claim 55, wherein said cancer is selected from the group consisting of a brain cancer, a head & neck cancer, an esophageal cancer, a lung cancer, a thyroid cancer, a stomach cancer, a colon cancer, a liver cancer, a kidney cancer, a prostate cancer, a breast cancer, a cervical cancer, an ovarian cancer, a testicular cancer, a rectal cancer, a skin cancer or a blood cancer.

62. The method of claim 55, wherein said composition is an expression cassette comprising a nucleic acid encoding one of said NSAID-modulated polypeptides, and a promoter active cells of said subject.

63. The method of claim 62, wherein said nucleic acid encodes an NSAID-stimulated polypeptides and is positioned sense to said promoter.

64. The method of claim 62, wherein said nucleic acid encodes an NSAID-inhibited polypeptides and is positioned anti-sense to said promoter.

65. The method of claim 62, wherein said expression cassette is incorporated in non-viral expression vector.

66. The method of claim 62, wherein said expression cassette is incorporated in viral expression vector.

67. The method of claim 66, wherein said viral expression vector is an adenoviral expression vector, a herpesviral expression vector, a retroviral expression vector, a vaccinia viral expression vector, an adeno-associated viral expression vector or a polyoma viral expression vector.

68. The method of claim 62, wherein said promoter is a constitutive promoter, a tumor specific promoter or an inducible promoter.

69. The method of claim 62, wherein said expression cassette further comprises a polyadenylation signal.

70. The method of claim 58, wherein said composition is an expression cassette comprising a nucleic acid encoding ribozyme that cleaves the mRNA encoding one of said NSAID-inhibited polypeptides, and promoter active cells of said subject.

71. The method of claim 58, wherein said composition is an expression cassette comprising a nucleic acid encoding single chain antibody that binds one of said NSAID-inhibited polypeptides, and promoter active cells of said subject.

72. The method of claim 55, wherein said composition is a small molecule.

73. The method of claim 55, further comprising treating said subject with chemotherapy, radiotherapy, immunotherapy or surgery.

74. The method of claim 55, further comprising monitoring the level of one or more NSAID-modulated polypeptides or transcript.

75. A method of screening compounds for NSAID-like activity comprising:
(a) providing a cell;
(b) contacting said cell with a candidate compound; and
(c) determining the candidate compound's effect on the expression of a plurality of NSAID-modulated genes,
wherein an effect similar to that seen in cells treated with known NSAIDs is predictive that said candidate compound exhibits NSAID-like activity.

76. The method of claim 75, wherein said plurality of NSAID-modulated genes are selected from the group consisting of the genes listed in Table 1 and Table 2.

77. The method of claim 76, wherein the plurality comprises at least one gene selected from the group consisting of the genes listed in Table 1 and Table 2.

78. The method of claim 77, wherein said plurality of genes comprises at least one gene selected from the group consisting of SEQ. ID. NO: 1, SEQ. ID. NO: 2, SEQ. ID. NO: 3, SEQ. ID. NO: 6, SEQ. ID. NO: 7, SEQ. ID. NO: 18, SEQ. ID. NO: 31, SEQ. ID. NO: 32, SEQ. ID. NO: 33, SEQ. ID. NO: 38, SEQ. ID. NO: 39, or SEQ. ID. NO: 42.

79. The method of claim 76, wherein the plurality comprises at least two genes selected from the group consisting of the genes listed in Table 1 and Table 2.

80. The method of claim 79, wherein the plurality comprises at least three genes selected from the group consisting of the genes listed in Table 1 and Table 2.

81. The method of claim 80, wherein the plurality comprises at least five genes selected from the group consisting of the genes listed in Table 1 and Table 2.

82. The method of claim 76, wherein the plurality comprises at least one gene that is not listed in Table 1 or Table 2.

83. The method of claim 75, wherein the plurality comprises at least one gene listed in Table 5.

84. The method of claim 75, further comprising determining the expression of a plurality of NSAID-modulated genes in the absence of said candidate compound.

85. The method of claim 75, further comprising determining the expression of a plurality of NSAID-modulated genes in a similar cell following treatment with a known NSAID.

86. The method of claim 75, wherein said cell is a cancer cell.

87. The method of claim 75, wherein determining comprises quantitating a mRNA for NSAID-modulated genes.

88. The method of claim 87, wherein quantitating comprises nucleic acid amplification.

89. The method of claim 87, wherein quantitating comprises, nucleic acid hybrizidation.

90. The method of claim 89, wherein hybridization comprises a nucleic acid array disposed on a chip or wafer.

91. The method of claim 87, wherein determining comprises quantitating NSAID-modulated polypeptides.

92. A nucleic acid array comprising a plurality of nucleic acid segments, each segment comprising at least 15 consecutive bases of a nucleic acid encoding a different NSAID-modulated polypeptide.

93. The nucleic acid array of claim 92, wherein said nucleic acid array is disposed on a chip or wafer.

94. The nucleic acid array of claim 92, wherein said plurality of NSAID-modulated genes are selected from the group consisting of the genes listed in Table I and Table 2.

95. The method of claim 94, wherein the plurality comprises at least one gene selected from the group consisting of the genes listed in Table 1 and Table 2.

96. The method of claim 95, wherein said plurality of genes comprises at least one gene selected from the group consisting of SEQ. ID. NO: 1, SEQ. ID. NO: 2, SEQ. ID. NO: 3, SEQ. ID. NO: 6, SEQ. ID. NO: 7, SEQ. ID. NO: 18, SEQ. ID. NO: 31, SEQ. ID. NO: 32, SEQ. ID. NO: 33, SEQ. ID. NO: 38, SEQ. ID. NO: 39, or SEQ. ID. NO: 42.

97. The method of claim 94, wherein the plurality comprises at least two genes selected from the group consisting of the genes listed in Table 1 and Table 2.

98. The method of claim 97, wherein the plurality comprises at least three genes selected from the group consisting of the genes listed in Table 1 and Table 2.

99. The method of claim 98, wherein the plurality comprises at least five genes selected from the group consisting of the genes listed in Table 1 and Table 2.

100. The method of claim 94, wherein the plurality comprises at least one gene that is not listed in Table 1 or Table 2.

101. The method of claim 92, wherein the plurality comprises at least one gene listed in Table 5.

102. A kit comprising a chip comprising a nucleic acid array comprising a plurality of nucleic acid segments, each segment comprising at least 15 consecutive bases of a nucleic acid encoding a different NSAID-modulated polypeptide.

103. The kit of claim 102, wherein said plurality of NSAID-modulated genes are selected from the group consisting of the genes listed in Table 1 and Table 2.

104. The method of claim 103, wherein the plurality comprises at least one gene selected from the group consisting of the genes listed in Table 1 and Table 2.

105. The method of claim 104, wherein said plurality of genes comprises at least one gene selected from the group consisting of SEQ. ID. NO: 1, SEQ. ID. NO: 2, SEQ. ID. NO: 3, SEQ. ID. NO: 6, SEQ. ID. NO: 7, SEQ. ID. NO: 18, SEQ. ID. NO: 31, SEQ. ID. NO: 32, SEQ. ID. NO: 33, SEQ. ID. NO: 38, SEQ. ID. NO: 39, or SEQ. ID. NO: 42.

106. The method of claim 103, wherein the plurality comprises at least two genes selected from the group consisting of the genes listed in Table 1 and Table 2.

107. The method of claim 108, wherein the plurality comprises at least three genes selected from the group consisting of the genes listed in Table 1 and Table 2.

108. The method of claim 107, wherein the plurality comprises at least five genes selected from the group consisting of the genes listed in Table I and Table 2.

109. The method of claim 103, wherein the plurality comprises at least one gene that is not listed in Table 1 or Table 2.

110. The method of claim 102, wherein the plurality comprises at least one gene listed in Table 5.
